(19) United States
(12) Patent Application Publication
(10) Pub. No.: US 2024/0081816 A1
(43) Pub. Date: Mar. 14, 2024

(54) BONE FIXATION TECHNIQUES AND IMPLANTS

(71) Applicant: Treace Medical Concepts, Inc., Ponte Vedra, FL (US)

(72) Inventors: W. Bret Smith, Durango, CO (US); Madeline Lindemann, Palm Coast, FL (US); Bryan Wilcox, St. Augustine, FL (US); Sean F. Scanlan, Jacksonville, FL (US); Jason May, St. John's, FL (US); Adriaan Kuyler, Ponte Vedra, FL (US); Robert D. Santrock, Morgantown, WV (US); Mark Erik Easley, Durham, NC (US)

(21) Appl. No.: 18/467,686

(22) Filed: Sep. 14, 2023

Related U.S. Application Data
(60) Provisional application No. 63/406,422, filed on Sep. 14, 2022.
(60) Provisional application No. 63/444,225, filed on Feb. 8, 2023.
(60) Provisional application No. 63/519,039, filed on Aug. 11, 2023.

(51) Int. Cl. A61B 17/064; A61B 17/10; A61B 17/88

(52) U.S. Cl. CPC A61B 17/0642; A61B 17/10; A61B 17/8897; A61B 2017/0641; A61B 2017/565

(57) ABSTRACT

An orthopedic implant system can be used to fixate two bones (e.g., to portions of a single bone) relative to each other during a surgical procedure. In some configurations, the implant system includes a staple having at least two legs separated by a bridge. The staple can include at least two couplings on either side of the bridge accessible through a top surface of the staple connectable to two corresponding coupling shafts. The coupling shafts can attach through the top surface of the staple without extending below an underside of the staple. The coupling shafts can be used to bias the at least two legs of the staple away from each other for insertion into holes formed into two bones. By attaching the coupling shafts through the top of the staple, the staple can be inserted flush with the two bones before releasing the shafts.

### RELATED CASES

This disclosure claims priority to U.S. provisional patent application No. 63/406,422, filed on Sep. 14, 2022; U.S. provisional patent application No. 63/444,225, filed on Feb. 8, 2023; and U.S. provisional patent application No. 63/519,039, filed on Aug. 11, 2023. The entire contents of each of these US provisional patent applications is hereby incorporated by reference.

### TECHNICAL FIELD

This disclosure generally relates to devices, systems, and techniques for fixating bones using one or more implants, including devices, systems, and techniques for fixating repositioned bones in the foot using one or more implants, such as a bone staple.

### BACKGROUND

Bones within the human body, such as bones in the foot, may be anatomically misaligned. For example, one common type of bone deformity is hallux valgus, which is a progressive foot deformity in which the first metatarsophalangeal joint is affected and is often accompanied by significant functional disability and foot pain. The metatarsophalangeal joint is laterally deviated, resulting in an abduction of the first metatarsal while the phalanges adduct. This often leads to development of soft tissue and a bony prominence on the medial side of the foot, which is called a bunion.

Surgical intervention may be used to correct a bunion deformity. A variety of different surgical procedures exist to correct bunion deformities and may involve removing the abnormal bony enlargement on the first metatarsal and/or realigning the first metatarsal relative to the adjacent metatarsal. In some procedures, an implant can be used to fixate a position of the metatarsal after realignment. The implant can be applied between the metatarsal and opposed cuneiform, across the tarsometatarsal joint. Such an implant can hold the metatarsal in a realigned position while bone grows to form a fused connection between the metatarsal and opposed cuneiform.

### SUMMARY

In general, this disclosure is directed to orthopedic implants as well as systems and surgical techniques for applying an implant to two opposed bones separated by a space between the bones. The space may be a joint, an osteotomy location, or a bone fracture that is being fused together. In some implementations, the one or more implants can be used to fixate a corrected position of a bone following a realignment procedure. Certain implant features and techniques disclosed herein can be useful to help to facilitate more efficient and effective bone fixation and resulting fusion. As one example, features relating to implants, implant systems, and implant techniques are disclosed herein that can help to increase the accuracy and/or stability of implant fixation relative to one or more target bones while also helping to decrease the time it takes to position and fixate the implant.

During a surgical procedure, an orthopedic implant can be applied to opposed bones across a space separating the bones, such as a joint separating different bones or a fracture or osteotomy separating a single bone into different portions. The implant can fixate the position of the bones relative to each other for healing during which bone growth closes the space between the bones, fusing the bones together. Example implants that can be used during an orthopedic procedures include a bone plate that is secured to underlying bones using two or more screws and/or a bone staple having at least two legs that are inserted into underlying bones.

In practice, it can be challenging for a clinician to align an implant with target anatomy where the implant is desirably positioned. This is particularly true when working on small bone anatomy, such as small bones in the foot. For example, during a surgical procedure, the clinician may seek to align the implant with the space separating the bones from each other. The clinician can position a first portion of the implant in and/or over one bone and a second portion of the implant in and/or over a second bone with the implant spanning the space between the bones (e.g., optionally substantially centering the length of the implant over the space). The clinician can drill holes in the bones where the implant is to be secured and then install the implant to the bones via the holes drilled in the bones. It can be challenging for the clinician to align the implant relative to the space between the bones and, when holes are drilled in the bone portions, to align the implant with the drill holes.

In accordance with some examples of the present disclosure, a surgical technique for installing an implant is described. The technique can involve inserting a first wire into a first bone and a second wire into a second bone, where the bones are separated from each other by a space. In some specific implementations, at least one of the bones is a metatarsal in the foot. For example, one of the bones may be a metatarsal and the other of the bones may be a cuneiform or cuboid separated by a tarsometatarsal joint or a phalanx separated by a metatarsophalangeal joint. In still another example, each of the bones may be portions of a metatarsal where the metatarsal is divided into two portions via an osteotomy and one portion (e.g., a distal portion) is realigned relative to the other portion (e.g., the proximal portion). In either case, at least one wire can be inserted into each respective bone to provide at least two wires. The wires can be parallel to each other, e.g., extending in the dorsal to plantar direction, and can be inserted through an incision or percutaneously through skin. One wire can be positioned on one side of the space between the two bones and the other wire positioned on the other side of the space.

With the wires positioned on opposite sides of the space, an implant operatively connected to an inserter can be guided along the wires to position the implant at a target location along the bones. The implant may be a plate, staple, or other implant configured to be placed in contact with external surfaces of the bones being fixated together. The inserter may have wire-receiving openings into which the wires inserted into the bones can be positioned. With the inserter aligned with the wires, the inserter can be guided along the wires until the implant contacts the underlying bones at a location set by the wires and the relative position of the implant to the inserter.

When the implant is a staple, the implant may be guided along the wires via the inserter until the legs of the staple are positioned in holes drilled in the underlying bone portions. Depending on the configuration of the inserter, the inserter may engage with and/or through a top surface of the staple without contacting the bottom surface of the staple (e.g., a bottom surface of the bridge of the staple). When so configured, the staple may be guided along the wires via the inserter and the staple positioned with the bottom surface contacting the bones before detaching the inserter from the staple. In some configurations, the inserter may be used to energize the staple (e.g., by applying a force to move the legs of the staple relative to each other for insertion followed by release of the force to cause the legs to bias back toward their native position).

Configuring the inserter and staple to allow the staple to be positioned with the legs of the staple in the bones and the bottom surface of the bridge contacting the external surfaces of the bones before disengaging the inserter from the staple can be useful to help ensure that the staple fully seats in the bones and establishes good fixation. While alternative inserter configurations can be used that engage the bottom surface of the staple (e.g., by including one or more legs that wrap around the staple), such an inserter configuration may necessitate removing the inserter from the staple to complete insertion of the staple into the bones. The inserter may be detached to remove the portion of the inserter wrapping around the staple from between the bottom surface of the staple and the bones. After the inserter is detached from the staple in such a configuration, however, the legs of the staple may bias back toward their natural position, resisting further insertion of the staple into the bones. This can make it more difficult for the clinician to complete insertion of the staple (e.g., requiring the clinician to pound the staple into the bones with the legs biased toward their natural position) as compared to an inserter configuration that allows the staple to be inserted to the full desired depth in the bones before disengaging the inserter.

When the implant is a bone plate, the implant may be guided along the wires via the inserter until the holes of the plate are positioned at target locations over the underlying bones (e.g., optionally aligned with holes drilled in the underlying bone portions). The clinician can insert screws through the holes of the bone plate to secure the bone plate to the underlying bones. The clinician can use one or more locking and/or compression screws.

When using guide wires to guide an inserter operatively connected to an implant, the wires can initially be positioned in the bones freehand without the aid of a guide or, in other examples, the clinician can use a guide to guide the introduction of the wires into the bones. The guide can have openings through which the wires can be placed into the underlying bones. The position of the openings in the guide can correspond to the position of the wire-receiving openings on the inserter (e.g., such that the wires inserted into the bones are at the same spacing and/or alignment as the wire-receiving openings on the inserter for subsequently engaging the inserter with the wires). In this way, the guide and inserter can work in combination to position wires in bones separated by a space and then guide an implant operatively connected to the inserter via the wires.

In some configurations, the guide includes and/or is couplable to a seeker that is insertable into the space between the two bones being fixated using the implant. To precisely and repeatably align an implant relative to the space, such as the tarsometatarsal joint space, the clinician can insert a seeker into the space. The seeker may be a wire or other elongated member insertable at least partially, and in some examples fully, into the space. The guide may include a seeker aperture that can be engaged with a portion of the seeker projecting out of the space between the bones. Alternatively, the guide may include a unitary or integral seeker (e.g., to provide a monolithic guide and seeker assembly) that is insertable into the space between the two bones. In either case, the seeker can align the guide with the space between the two bones being fixated. In turn, the guide can have one or more wire alignment features (e.g., one or more openings) usable to introduce a wire into the first bone at a designated location spaced from the seeker and a wire into the second bone at a designated location spaced from the seeker. Accordingly, the guide can set the position of the wires and, correspondingly, the resulting position of the implant, relative to the space between the two bones.

In some configurations, the guide is used only to align with the space between the two bones and/or to insert wires in the two bones for subsequently guiding positioning of an implant. In other configurations, the guide may additionally or alternatively be used to guide drilling of one or more implant holes in one or both bones being fixated using the implant. For example, the guide may include one or more drill apertures positionable over a first bone being fixated and one or more drill apertures positionable over a second bone being fixated. The clinician can insert a bone removal tool through the drill apertures to form corresponding cavities in the underlying bones. The cavities can be configured (e.g., sized, shaped, positioned) to receive fixation members associated with the implant, such as legs of the staple or screws associated with the bone plate. The location of the cavities formed in the bones can be at designated locations relative to the wires previously or subsequently inserted in bones and/or the space between the bones located by the seeker. Accordingly, when an inserter operatively coupled to an implant is subsequently guided along the wires, the fixation features of the implant can be accurately aligned with the cavities formed in the bones.

In some examples according to the disclosure, an orthopedic implant is described in the form of a staple. The staple can be applied to a metatarsal bone and a cuneiform bone across a tarsometatarsal joint, for instance during a metatarsal fusion procedure. The staple can be applied to different bones or bone portions (e.g., across a joint, fracture, or osteotomy), as described herein. In one example, the staple can be applied to a first metatarsal and a medial cuneiform across a first tarsometatarsal joint. In another example, the staple can be applied to a metatarsal and a proximal phalanx across a metatarsophalangeal joint. As a further example, the staple can be applied to a proximal portion and a distal portion of a metatarsal across an osteotomy or fracture dividing the metatarsal into the proximal portion and the distal portion.

The staple can be configured to transition between a natural, undeformed state and a deformed insertion state upon application/removal of a load force. As such, when the staple is being inserted into the first and second bones, the load force can be applied to the staple to cause the staple to transition from the natural, undeformed state to the deformed insertion state. After the staple has been desirably inserted into the first and second bones, the load force can be removed from the staple to cause the staple to transition from the deformed insertion state to a compression-inducing state. With the staple positioned in a first bone (e.g., a metatarsal) and a second bone (e.g., a cuneiform) across a space between the first and second bones (e.g., a tarsometatarsal joint space) and transitioned to a compression-inducing state, the staple can act to apply a compression force on each of the first bone and the second bone to compress the bones together across the space.

In one example, a method of fixating bones for fusion is described. The method includes inserting a first wire into a first bone and a second wire into a second bone, with the first bone being separated from the second bone by a space. This method also includes aligning an inserter operatively connected to an implant with the first wire and the second wire by at least positioning the first wire in a first wire receiving opening of the inserter and the second wire in a second wire receiving opening of the inserter. This method includes advancing the inserter along the first wire and the second wire to position the implant in contact with the first bone and the second bone with the implant bridging across the space between the first bone and the second bone.

In another example, a method of fixating bones for fusion is described. The method includes positioning a first leg of a staple connected to an inserter in a first implant hole of a first bone and a second leg of the staple connected to the inserter in a second implant hole of a second bone. The first bone is separated from the second bone by a space and the first leg of the staple is connected to the second leg of the staple by a bridge. According to the example, the inserter includes a first coupling shaft connected to the first leg of the staple, a second coupling shaft connected to the second leg of the staple, and a connector. The first coupling shaft and the second coupling shaft are biased toward each other to apply a load force to the implant. The connector joins the first coupling shaft and the second coupling shaft to maintain the load force applied to the implant while the connector joins the first coupling shaft and the second coupling shaft. The method also includes removing the connector from the inserter, thereby removing the load force and causing the first coupling shaft and the second coupling shaft to move apart from one another.

In a further example, a system for fixating bones for fusion is described. The system includes a staple and an inserter. The staple includes a first leg on a first side of the staple, a second leg on a second side of the staple, a bridge connecting the first leg and the second leg, a first handling coupling on the first side of the staple, and a second handling coupling on the second side of the staple. The inserter includes a first coupling shaft configured to couple to the first handling coupling, a second coupling shaft configured to couple to the second handling coupling, and a connector configured to join the first coupling shaft and the second coupling shaft. The connector includes a first wire receiving opening configured to receive a first wire and a second wire receiving opening configured to receive a second wire. When the connector is joined to the first coupling shaft and the second coupling shaft, the connector is configured to bias the first coupling shaft and the second coupling shaft toward each other to apply a load force to the staple. When the connector is removed from at least one of the first coupling shaft and the second coupling shaft, the staple is configured such that the first leg and the second leg move toward one another.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIG. 4 is a flow diagram of an example surgical technique for positioning and fixating an implant.

FIGS. 5A and 5B illustrate an orthopedic implant in the form of a staple. FIG. 5A is a perspective view of this embodiment of the staple, and FIG. 5B is a side elevational view of a longitudinal cross-section of the staple shown at FIG. 5A.

FIG. 6 is a perspective view of another example configuration of a staple.

FIG. 7 is a perspective view of another example configuration of a staple.

FIG. 8 is a perspective view of another example configuration of a staple.

FIGS. 9A and 9B illustrate an embodiment of a guide. FIG. 9A is a perspective view of this guide, and FIG. 9B is a top plan view of a body of the guide shown at FIG. 9A.

FIGS. 10A and 10B illustrate another embodiment of a guide. FIG. 10A is a perspective view of this guide, and FIG. 10B is a top plan view of a body of the guide shown at FIG. 10A.

FIGS. 11A-11C illustrate an embodiment of an inserter.

FIGS. 12A and 12B illustrate another embodiment of an inserter. FIG. 12A shows a side elevational view of a longitudinal cross-section of this inserter, and FIG. 12B shows a bottom plan view of a cap of this inserter.

FIGS. 13A and 13B illustrate yet another embodiment of an inserter. FIG. 13A shows a perspective view of this inserter, and FIG. 13B shows a bottom plan view of a cap of this inserter.

FIG. 14 is a perspective view of an implant tamp feature that can be included at one or more of the inserter embodiments shown at FIGS. 11A-13B.

FIG. 15 is a flow diagram of an example surgical technique for positioning an implant, such as a staple, to fixate bones for fusion.

FIG. 16 is a side elevational view of an embodiment of a seeker inserted into a space (e.g., a joint space) separating first and second bones.

FIG. 17 is a side elevational view of an embodiment of a guide aligned with a first and/or a second bone using the seeker of FIG. 16.

FIG. 18 is a side elevational view of the guide of FIG. 17 used to drill a first implant hole in a first bone and a second implant hole in a second bone.

FIG. 19A-19C illustrate an embodiment of an inserter that is to be aligned with wire(s) at the first and/or second bones. FIG. 19A is an elevational view of the inserter coupled to a top side of an implant, FIG. 19B is a perspective view of a connector component of the inserter in isolation, and FIG. 19C is an elevational view of wires at the first and second bones for aligning the inserter.

FIG. 20 is a side elevational view of the assembled inserter of FIGS. 19A and 19B advanced along the wire(s) to position an implant, such as a staple, in contact with the first and second bones.

FIG. 21 is a side elevational view of the inserter of FIG. 20 with a connector component removed such that the implant helps to fixate the first and second bones and applies a compression force at the first and second bones.

FIGS. 22A and 22B illustrate another embodiment of an orthopedic implant in the form of a staple that includes fixation apertures. FIG. 22A is a perspective view of this embodiment of the staple showing the fixation apertures, and FIG. 22B is a perspective view of this embodiment of the staple showing fixation members placed at the fixation apertures.

FIGS. 23A and 23B illustrate an additional embodiment of an orthopedic implant in the form of a staple that includes fixation apertures. FIG. 23A is a perspective view of this embodiment of the staple showing the fixation apertures, and FIG. 23B is a perspective view of this embodiment of the staple showing fixation members placed at the fixation apertures.

FIGS. 24A and 24B illustrate a further embodiment of an orthopedic implant in the form of a staple that includes fixation apertures as well as certain exemplary anatomical contouring. FIG. 24A is a side elevational view of this embodiment of the staple showing exemplary anatomical contouring, and FIG. 24B is a side elevational view showing such staple embodiments with exemplary anatomical contouring placed across a space (e.g., a joint space) separating first and second bones.

FIGS. 25A and 25B illustrate another embodiment of an orthopedic implant in the form of a staple that includes fixation apertures as well as certain exemplary anatomical contouring. FIG. 25A is a side elevational view of this embodiment of the staple showing exemplary anatomical contouring, and FIG. 25B is a side elevational view showing such staple embodiment with exemplary anatomical contouring placed across a space (e.g., a joint space) separating first and second bones.

FIGS. 26A and 26B illustrate an embodiment of a multi-piece inserter coupling shaft. FIG. 26A is an exploded, elevational view of this inserter coupling shaft embodiment, and FIG. 26B is an assembled view of this inserter coupling shaft embodiment.

FIGS. 27A-27D illustrate another embodiment of a multi-piece inserter coupling shaft. FIG. 27A is an exploded, elevational view of this inserter coupling shaft embodiment.

FIG. 27B is a close-up elevational view of a distal end portion of this inserter coupling shaft embodiment. FIG. 27C is an elevational view of this inserter coupling shaft embodiment coupled to an exemplary orthopedic implant. FIG. 27D is a close-up elevational view of a distal end portion of this inserter coupling shaft embodiment when coupled to the exemplary orthopedic implant as in FIG. 27C.

FIGS. 28A-28C illustrate another embodiment of an orthopedic implant in the form of a staple. FIG. 28A is a longitudinal side elevational view of this embodiment of the staple, FIG. 28B is a radial side elevational view of this embodiment of the staple, and FIG. 28C is a top plan view of this embodiment of the staple.

Like reference characters are used in the following description and in the drawings to indicate like elements.

### DETAILED DESCRIPTION

This disclosure generally relates to implants for fixating one or more bones, associated systems (e.g., kits) for bone fixation, and bone fixation techniques incorporating one or more such implants. In some examples, an implant according to this disclosure can be used to fixate a repositioned bone, or bones, during a surgical procedure, such as a metatarsal realignment and fusion procedure. In exemplary applications, the devices, systems, and techniques can be used during a surgical procedure performed on one or more bones, such as a bone alignment, osteotomy, fusion procedure, fracture repair, and/or other procedures where one or more bones are to be set in a desired position. Such a procedure can be performed, for example, on bones (e.g., adjacent bones separated by a joint or different portions of a single bone) in the foot or hand, where bones are relatively small compared to bones in other parts of the human anatomy. In one example, a procedure utilizing devices and/or techniques of the disclosure can be performed to correct an alignment between a metatarsal (e.g., a first metatarsal) and a cuneiform (e.g., a medial cuneiform), such as a bunion correction. An example of such a procedure is a lapidus procedure. In another example, the devices, systems, and/or techniques can be utilized when modifying a position of one portion of a bone relative to another portion of the same bone. An example of such a procedure is an osteotomy procedure (e.g., metatarsal osteotomy procedure) in which the bone is cut into at least two different bones and one portion (e.g., a distal portion) is realigned relative to another bone portion (e.g., a proximal portion) of the same bone.

Preparation, fixation, and/or fusion of two opposed bone portions, such as a metatarsal and cuneiform, may be performed according to the disclosure for a variety of clinical reasons and indications. Preparation and fusion of a metatarsal and cuneiform at the tarsometatarsal (“TMT”) joint may be performed to treat hallux valgus and/or other bone and/or joint conditions.

Hallux valgus, also referred to as hallux abducto valgus, is a complex progressive condition that is characterized by lateral deviation (valgus, abduction) of the hallux and medial deviation of the first metatarsophalangeal joint. Hallux valgus typically results in a progressive increase in the hallux abductus angle, the angle between the long axes of the first metatarsal and proximal phalanx in the transverse plane. An increase in the hallux abductus angle may tend to laterally displace the plantar aponeurosis and tendons of the intrinsic and extrinsic muscles that cross over the first metatarsophalangeal joint from the metatarsal to the hallux. Consequently, the sesamoid bones may also be displaced (e.g., laterally relative to the first metatarsophalangeal joint), resulting in subluxation of the joints between the sesamoid bones and the head of the first metatarsal. This can increase the pressure between the medial sesamoid and the crista of the first metatarsal head.

While techniques and devices are generally described herein in connection with the first metatarsal and medial cuneiform of the foot, the techniques and devices may be used on other adjacent bones (e.g., separated from each other by a joint) and/or adjacent bone portions (e.g., portions of the same bone separated from each other by a fracture or osteotomy). In various examples, the devices, systems, and/or techniques of the disclosure may be utilized on comparatively small bones in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof. The bones may be separated from each other by a tarsometatarsal (“TMT”) joint, a metatarsophalangeal (“MTP”) joint, or other joint. Accordingly, reference to a first metatarsal and medial cuneiform herein may be replaced with other bone pairs as described herein. Further, where an implant according to the disclosure is intended to be used on a different bone or combination of bones other than the first metatarsal and medial cuneiform, the configuration of the implant (e.g., size, shape) may be adjusted to accommodate the specific bone or combination of bones being fixated while following the implant, such as staple, configuration teachings outlined herein.

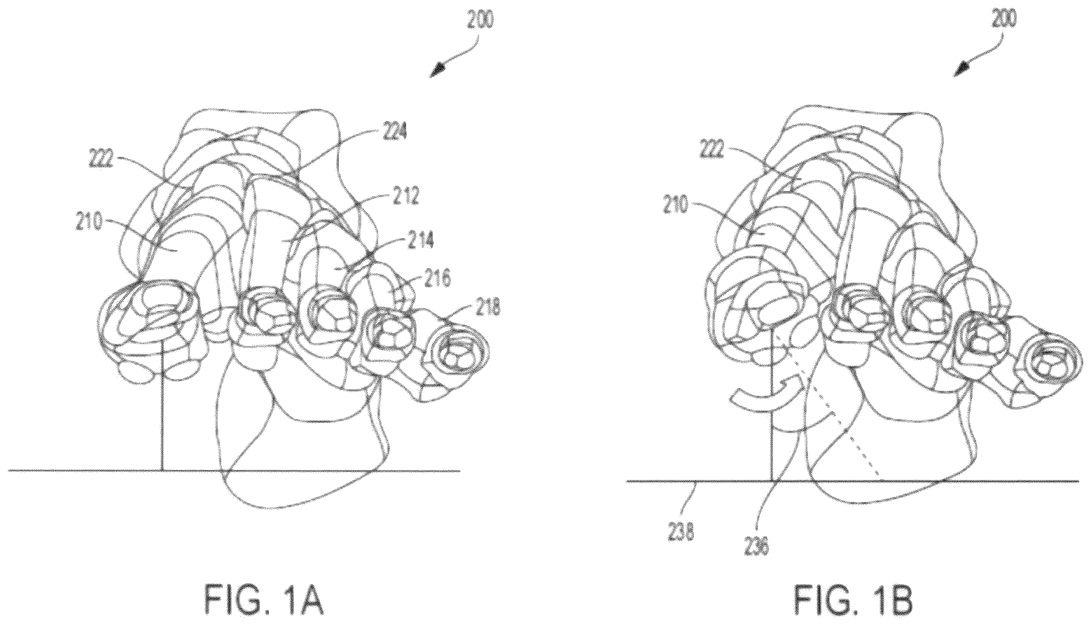

To further understand example devices, systems, and techniques of the disclosure, the anatomy of the foot will first be described with respect to FIGS. 1-3 along with example misalignments that may occur and be corrected and fixated according to the present disclosure. A bone misalignment may be caused by hallux valgus (bunion), a natural growth deformity, metatarsus adductus, arthritis, and/or other condition.

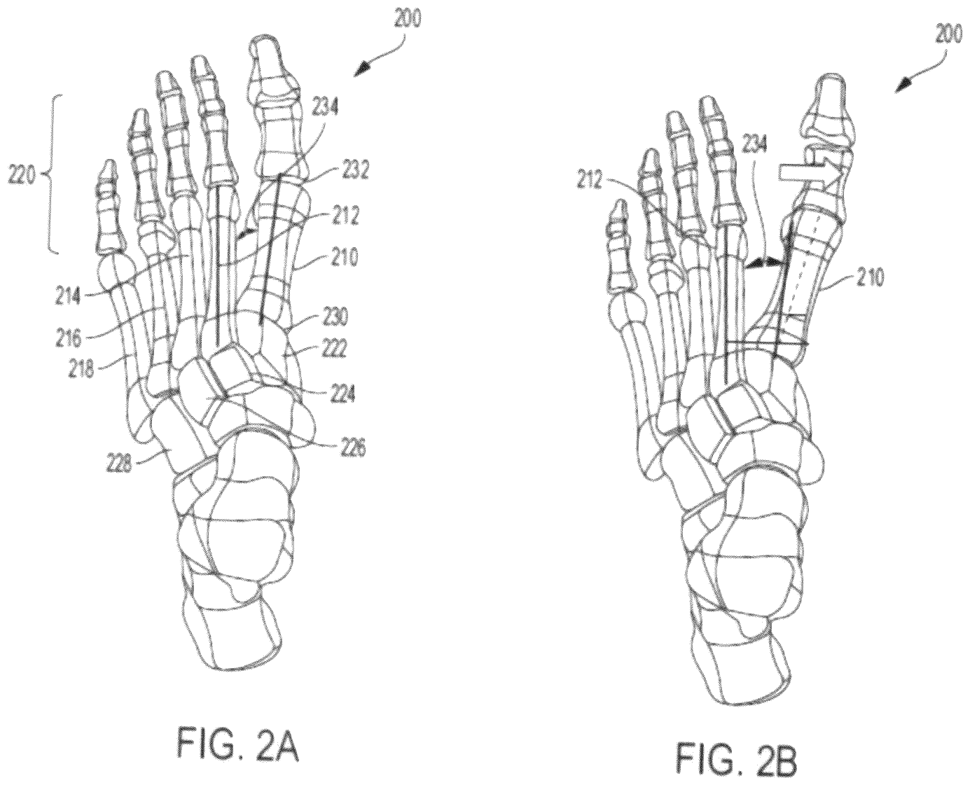

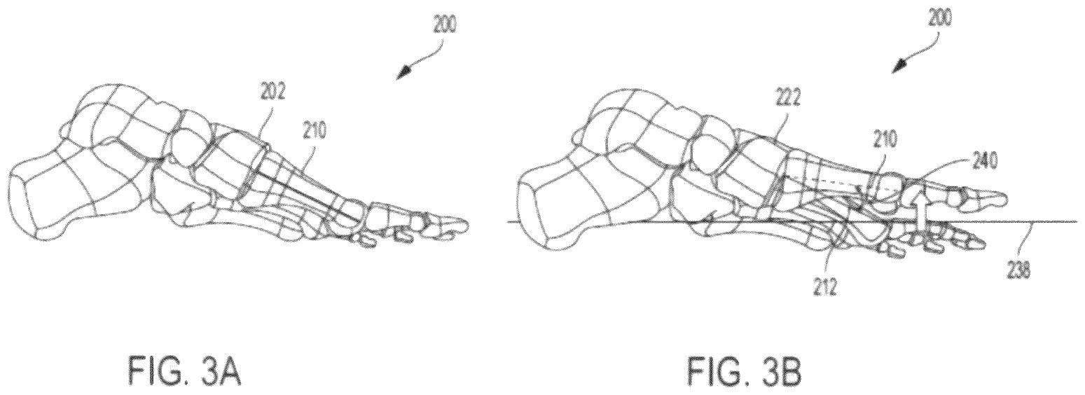

FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected. Further, a bone condition treated according to the disclosure may not present any of the example misalignments described with respect to FIGS. 1B, 2B, and 3B, and it should be appreciated that the disclosure is not limited in this respect.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. The first metatarsal 210 is connected proximally to a medial cuneiform 222, while the second metatarsal 212 is connected proximally to an intermediate cuneiform 224 and the third metatarsal 214 is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The joint 232 between a metatarsal and respective proximal phalanx is referred to as a metatarsophalangeal joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and is perpendicular to an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

Surgical techniques and instruments according to the disclosure can be useful during a procedure to correct a misalignment of one or more bones, such as the metatarsal and opposed cuneiform, and/or to promote fusion of the metatarsal and cuneiform across the TMT joint. In some applications, a realignment procedure involves surgically accessing the TMT joint (e.g., from a medial side of the foot and/or a dorsal side of the foot). The clinician can insert a bone preparation instrument through an incision to prepare the end face of one or both bones.

Before or after preparing one or both ends of first metatarsal 210 and medial cuneiform 222, the clinician can realign the metatarsal relative to the cuneiform. The clinician can pivot the distal end of first metatarsal 210 laterally toward second metatarsal 212 to close an intermetatarsal angle between the first and second metatarsal. Additionally or alternatively, the clinician can rotate first metatarsal 210 in the frontal plane to correct a frontal plane rotation of the metatarsal and/or move the first metatarsal 210 in the sagittal plane to correct a sagittal plane position of the metatarsal. Realignment of first metatarsal 210 can be performed freehand by the clinician or with the aid of a bone positioning device to facilitate the realignment. After desired realignment in one or more planes, the clinician can fixate the moved position of first metatarsal 210 by applying one or more implants (e.g., one or more staples, plates, pins, screws, rods). The present disclosure provides exemplary embodiments of a staple as one type of implant that can be used to fixate one or more bones for fusion, though other embodiments within the scope of this disclosure can use the teachings outlined herein applied to other types of implants, such as a plate, for fixating one or more bones for fusion.

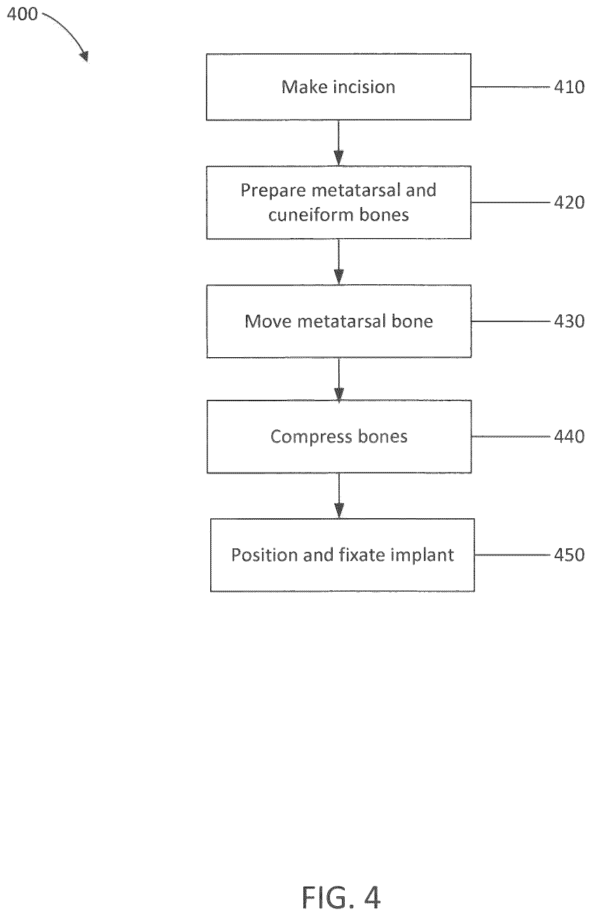

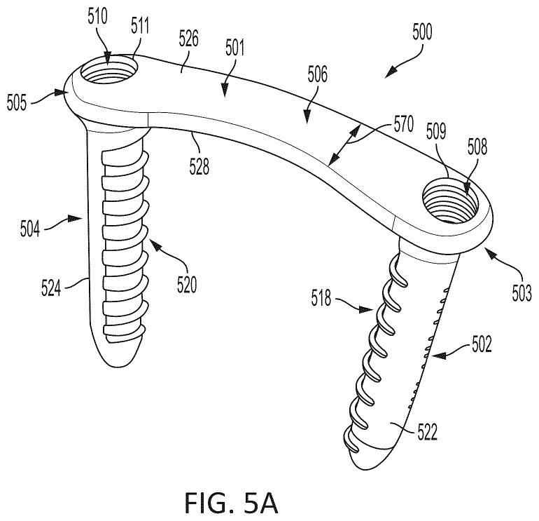

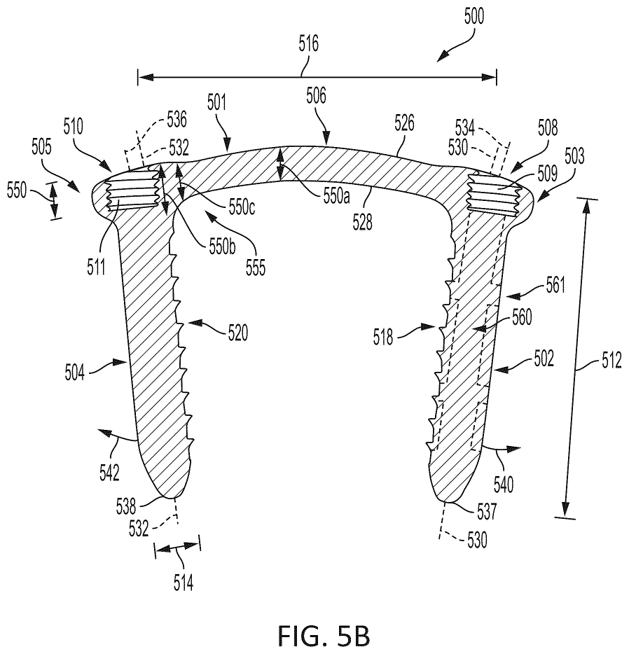

FIG. 4 is a flow diagram of an example method 400 that can include, among other steps, positioning and fixating an implant to fixate bones for fusion. As will be described, in one example the method 400 can be used to prepare, realign, and fixate two bones across a tarsometatarsal joint. One or more portions of the method 400 relating to positioning and fixating an implant to fixate bones for fusion (step 450) will be described further with reference to FIGS. 5-21. Additional details on example surgical techniques, including example instrumentation that can be used during the techniques, can be found in U.S. Pat. No. 9,622,805, issued Apr. 18, 2017 and entitled “BONE POSITIONING AND PREPARING GUIDE SYSTEMS AND METHODS” and US Patent Publication No. 2020/0015856, published Jan. 16, 2020 and entitled “COMPRESSOR-DISTRACTOR FOR ANGULARLY REALIGNING BONE PORTIONS,” the entire contents of each of which are incorporated herein by reference.

At step 410, the method 400 includes making an incision. The incision can be made through the skin, such as on a dorsal side of the foot, a medial side of the foot, or on a dorsal-medial side of the foot. The incision can be made to provide surgical access to the TMT joint 230 which separates first metatarsal 210 from opposed medial cuneiform 222. To surgically access the joint, the patient may be placed in a supine position on the operating room table and general anesthesia or monitored anesthesia care administered. Hemostasis can be obtained by applying thigh tourniquet or mid-calf tourniquet. In some examples, imaging of the foot can be used to assist the clinician in ascertaining the location of TMT joint 230 about which incision can be centered when subsequently cutting through skin.

At step 420, the method 400 includes preparing first metatarsal 210 and/or medial cuneiform 222. With the TMT joint 230 exposed via the incision, an end face (e.g., proximal end face) of first metatarsal 210 and/or an end face (e.g., distal end face) of medial cuneiform 222 can be prepared. It is to be noted that one or both of the end faces of the metatarsal and the cuneiform can be prepared before and/or after the metatarsal is moved relative to the cuneiform in one or more planes. Accordingly, unless otherwise specified, the order of bone preparation and/or movement is not limited.

In general, the clinician can prepare the end of each bone forming TMT joint 230 so as to promote fusion of the bone ends across the TMT joint following realignment. Bone preparation may involve using a tissue removing instrument to apply a force to the end face of the bone so as to create a bleeding bone face to promote subsequent fusion. Example tissue removing instruments that can be used include, but are not limited to, a saw, a rotary bur, a rongeur, a reamer, an osteotome, a curette, and the like. The tissue removing instrument can be applied to the end face of the bone being prepared to remove cartilage and/or bone. For example, the tissue removing instrument may be applied to the end face to remove cartilage (e.g., all cartilage) down to subchondral bone. Additionally or alternatively, the tissue removing instrument may be applied to cut, fenestrate, morselize, and/or otherwise reshape the end face of the bone and/or form a bleeding bone face to promote fusion. In instances where a cutting operation is performed to remove an end portion of a bone, the cutting may be performed freehand or with the aid of a cutting guide having a guide surface positionable over the portion of bone to be cut. When using a bone preparation guide, a cutting instrument can be inserted against a guide surface (e.g., between a slot define between two guide surfaces) of the bone preparation guide to guide the cutting instrument for bone removal.

At step 430, the method 400 includes moving first metatarsal 210. As noted, first metatarsal 210 can be moved before and/or after first metatarsal 210 and/or medial cuneiform 222 are prepared. Moving first metatarsal 210 at step 430 can include moving first metatarsal 210 in at least one plane. For example, first metatarsal 210 can be moved in at least transverse plane to close IMA 234 between first metatarsal 210 and adjacent second metatarsal 212 and/or a frontal plane (e.g., to reposition the sesamoid bones substantially centered under the metatarsal). In some examples, first metatarsal 210 can be moved in multiple planes, such as the transverse plane and/or frontal plane and/or sagittal plane (e.g., each of the transverse, frontal, and sagittal planes). The clinician may or may not utilize a bone positioning device to facilitate movement of the bone portion. The moved position of first metatarsal 210 can result is realignment of first metatarsal 210 relative to one of more other adjacent bones.

At step 440, the method 400 may include compressing one or more bones. In some embodiments, the step 440 can be omitted depending on the realigned position of the first metatarsal 210. When step 440 is included, the prepared end faces of the bone portions of first metatarsal 210 and medial cuneiform 222 can be compressed together prior to fixating bones using an implant. The clinician may compress the end faces together with hand pressure and/or using a compressing instrument physically attached to both the first bone portion and the second bone portion. Accordingly, discussion of applying an implant to a first bone portion and a second bone portion, with the implant bridging a space between the bone portions, refers to the implant bridging a separation (e.g., joint, osteotomy, fracture) between the two bone portions but does not require a gap between the bone portions, as the end faces of the bone portions may be in contact with each other and compressed together.

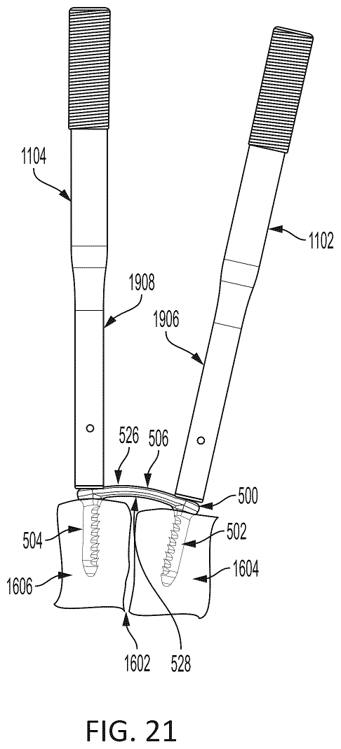

At step 450, the method 400 includes positioning an implant (e.g., an embodiment of a staple disclosed herein) over a portion of first metatarsal 210 and over a portion of medial cuneiform 222 and across TMT joint 230 separating first metatarsal 210 from medial cuneiform 222. For example, as shown at FIG. 21, positioning an implant at step 450 can include positioning an embodiment of a staple (e.g., compression staple). Positioning an implant, such as a staple, can include positioning at least one leg of the staple over first metatarsal 210 and positioning at least another leg of the staple over medial cuneiform 222 with a bridge, which connects the legs of the staple, extending across the TMT joint 230. Further, in some examples, positioning an implant, such as a staple, can include positioning one leg of the staple over first metatarsal 210 and then into contact with first metatarsal 210 in a first implant hole at first metatarsal 210 and positioning another leg of the staple over medial cuneiform 222 and then into contact with medial cuneiform 222 in a second implant hole at medial cuneiform 222.

FIGS. 5A-8 illustrate various embodiments of a staple as one exemplary type of implant that can be used to fixate bones for fusion.

FIGS. 5A and 5B illustrate an embodiment of a staple 500. FIG. 5A is a perspective view of the staple 500, and FIG. 5B is a side elevational view of a longitudinal cross-section of the staple 500. As described elsewhere herein, the staple 500 can be configured to apply a compression force at the bones and across the space (e.g., joint) between the bones for use in fixating and promoting fusion of bones.

The staple 500 can include a staple body 501 having a first leg 502, a second leg 504, and a bridge 506. For the illustrated embodiment, the staple 500 includes the first leg 502 at a first side 503 of the staple 500 and the second leg 504 at a second side 505 of the staple 500. In this example, the first side 503 is opposite the second side 505. The bridge 506 can connect the first leg 502 and the second leg 504.

The legs 502, 504 of the staple 500 can be configured for positioning in bones, such as one or more relatively small bones of the foot. For example, a length 512 of each of the legs 502, 504 can be within a range from 6 mm to 30 mm, such as from 8 mm to 25 mm or from 10 mm to 20 mm, which can provide sufficient length to robustly anchor within a bone of the foot, such as a metatarsal (e.g., first metatarsal) and/or cuneiform (e.g., medial cuneiform). A width 514 of each of the legs can be within a range from 1 mm to 4 mm, such as from 1.5 mm to 3.5 mm or from 2 mm to 3 mm, which likewise can provide sufficient length to robustly anchor within a bone of the foot. A bridge length 516 of the bridge 506 can be within a range from 6 mm to 30 mm, such as from 8 mm to 25 mm, or from 12 mm to 20 mm, which can be sufficient to allow for positioning the bridge across a space (e.g., joint) between bones in the foot while maintaining the legs 502, 504 at such bones separated by the space. A staple according to the disclosure can be configured with dimensions other than the foregoing examples, and the disclosure is not limited in this respect.

As an example shown at FIG. 5B, the bridge length 516 can be as measured from a central longitudinal axis of one leg closest to one side of the bridge 506 to a central longitudinal axis of another leg closest to an opposite side of the bridge 506. The bridge 506 can also define a bridge width 570. In some examples, the bridge width 570 of the staple 500 is constant over the entire width of the staple. In other examples, the bridge width 570 of the staple 500 varies over the width of the staple. For example, the bridge width 570 can be substantially constant over a central region along the bridge length 516, and the bridge width 570 can increase moving along the bridge length 516 from the central region toward each of the first leg 502 and the second leg 504 (e.g., such that the greatest bridge width 570 is at or adjacent the first and second legs 502, 504). As one specific such example, the bridge width 570 can define a generally hourglass profile with the narrower, center of the hourglass at a central region along the bridge length 516 and the increasing width end portions of the hourglass at opposite ends of the bridge length 516.

The first leg 502 can include a first set of teeth 518 at a perimeter 522 of the first leg 502, and the second leg 504 can include a second set of teeth 520 at a perimeter 524 of the second leg 504. The teeth 518, 520 can extend out from the respective leg 502, 504 and be configured to provide an anchoring mechanism for maintaining the respective leg 502, 504 within the respective bone at which the respective leg 502, 504 is placed. As illustrated here, the first set of teeth 518 can extend partially around the perimeter 522 of the first leg 502, and the second set of teeth 520 can extend partially around the perimeter 524 of the second leg 504. For example, the first set of teeth 518 can extend around a portion of the perimeter 522 of the first leg 502 facing the bridge 506, and the second set of teeth 520 can extend around a portion of the perimeter 525 of the second leg 504 facing the bridge 506. As one specific such example, the first set of teeth 518 can extend around approximately one hundred and eighty degrees of the perimeter 522 of the first leg 502 nearest the bridge 506, and the second set of teeth 520 can extend around approximately one hundred and eighty degrees of the perimeter 524 of the second leg 504 nearest the bridge 506.

The staple 500 can further include a first handling coupling 508 and a second handling coupling 510 defined by the staple body 501. For the illustrated embodiment, the staple 500 includes the first handling coupling 508 at the first side 503 of the staple 500 and the second handling coupling 510 at the second side 505 of the staple 500. The first handling coupling 508 can include a first handling coupling receptacle 509 extending from a top surface 526 of the staple body 501 of the staple 500 toward (e.g., to) a bottom surface 528 of the staple body 501 of the staple 500. As one such specific example, the first handling coupling receptacle 509 can extend from the top surface 526 down a portion, but less than all of, the length 512 of the first leg 502. The second handling coupling 510 can include a second handling coupling receptacle 511 extending from the top surface 526 of the staple body 501 of the staple 500 toward (e.g., to) the bottom surface 528 of the staple body 501 of the staple 500. As one such specific example, the second handling coupling receptacle 511 can extend from the top surface 526 down a portion, but less than all of, the length 512 of the second leg 504. As such, the first handling coupling 508 and first handling coupling receptacle 509 as well as the second handling coupling 510 and second handling coupling receptacle 511 can be accessible from the top surface 526 of the staple 500, which can be useful in helping to facilitate generally flush placement of the bottom surface 528 of the staple 500 against one or more bones (e.g., against each of two bones separated by a space, such as a joint).

The first handling coupling receptacle 509 of the first handling coupling 508 can be configured to couple to a first coupling shaft of an inserter, such as at a location between the top surface 526 and a bottom surface of the handling coupling receptacle. The second handling coupling receptacle 511 of the second handling coupling 510 can be configured to couple to a second coupling shaft of an inserter, such as at a location between the top surface 526 and a bottom surface of the handling coupling receptacle. As such, the first and second handling coupling receptacles 509, 511 can be configured to operatively couple to the respective first and second coupling shafts of the inserter such that the first and second coupling shafts of the inserter are inserted into the respective first and second handling coupling receptacles 509, 511 from the top surface 526 and maintained within the respective first and second handling coupling receptacles 509, 511 so as to not extend out from the bottom surface of the handling coupling receptacles. The bottom end of each handling coupling receptacle can be closed with a solid portion first leg 502 or second let 504, respectively, extending beyond the bottom end of the handling coupling receptacle.

In the illustrated example of FIGS. 5A and 5B, the first and second handling coupling receptacles 509, 511 includes threads extending along a length of the first and second handling coupling receptacles 509, 511 between the top and bottom surfaces of the handling coupling receptacle. These threads can be configured to connect to complementary threads on respective first and second coupling shafts of an inserter. Though in other embodiments the first and second handling coupling receptacles 509, 511 and first and second coupling shafts of the inserter can include others means to facilitate an operative connection therebetween the respective components (e.g., a bayonet connection).

Depending on the application in which the staple 500 is used, the staple 500 can be configured to receive one or more solid or liquid substances after insertion of the staple into bone. As one such example, one or both of the first and second handling coupling receptacles 509, 511 can be configured to receive a filler material therein to substantially plug the first and/or second handling coupling receptacles 509, 511 at the top surface 526. This filler material can be placed in the first and/or second handling coupling receptacles 509, 511 after removing the respective first and/or second coupling shaft from the respective first and/or second handling coupling receptacles 509, 511. For instance, a biologically compatible wax or other biologically compatible filler material can be placed into the first and/or second handling coupling receptacles 509, 511 to plug the first and/or second handling coupling receptacles 509, 511 at or near the top surface 526 so as to help impede bone ingrowth and/or passage of biologic substances into the first and/or second handling coupling receptacles 509, 511 after removing corresponding inserter coupling shafts.

As another such example, one or both of the legs 502, 504 can be configured to receive and convey a substance therethrough. For instance, a cannula 560 can be defined within one or both of the legs 502, 504. When included, the cannula 560 can extend along at least a portion (e.g., all) of the length 512 of the leg 502 and/or 504. In one example, the cannula 560 can extend coaxial with a coupling shaft received at a handling coupling (e.g., handling coupling 508) at the staple 500. When included, the cannula 560 can have an inlet, for instance at the bottom end of the respective handling coupling 508, 510, and the cannula 560 can have one or more outlets 561 at a location along the respective leg 502, 504 spaced apart from the respective handling coupling 508, 510. For the illustrated embodiment, the outlets 561 can be included at the respective leg 502 and/or 504 between teeth 518, 520. A medication, structural support substance, or other biologically compatible substance can be introduced into the cannula 560 at the inlet (e.g., at the respective handling coupling 508, 510) and this substance can be delivered to one or more bones, at which the staple 500 is placed, via the one or more outlets 561. In other examples, staple 500 does not include a cannula 560 extending through either leg 502, 504. Rather, each leg may be solid body with a respective handling coupling 508, 510 formed extending partially but not fully down the length of the solid body without a further cannulation extending through the leg.

Additionally or alternatively, the staple 500 can be configured with a cannulation extending through the length of leg 502 and/or 504 for receiving corresponding wires inserted into bone to help facilitate positioning and placement of the staple into underlying bone. For example, in lieu of using an inserter having wire receiving openings to guide positioning of an implant as will be described, wires inserted into underlying bones can be aligned with cannulations extending through at least two legs of the staple. The cannulations can be aligned with the wires positioned in the bones and the staple guided along the wires.

The first leg 502 can define, and length 512 of the leg can extend along, a first leg central longitudinal axis 530 extending through a geometric center of the leg. The second leg 504 can define, and the length 512 of the leg can extend along, a second leg central longitudinal axis 532 extending through a geometric center of the leg. Likewise the first handling coupling receptacle 509 can define and extend a length from the top surface 526 toward (e.g., to) a bottom surface along a first handling coupling receptacle central longitudinal axis 534 that extends through a geometric center of the handling coupling receptacle. The second handling coupling receptacle 511 can define and extend a length from the top surface 526 toward (e.g., to) a bottom surface along a second handling coupling receptacle central longitudinal axis 536 that extends through a geometric center of the handling coupling receptacle.

As shown for the illustrated embodiment of the staple 500 at FIG. 5B, the first leg central longitudinal axis 530 can be offset from the first handling coupling receptacle central longitudinal axis 534, and the second leg central longitudinal axis 532 can be offset from the second handling coupling receptacle central longitudinal axis 536. In particular, in this illustrated embodiment of the staple 500, the first leg central longitudinal axis 530 can be closer to the bridge 506 than the first handling coupling receptacle central longitudinal axis 534, and the second leg central longitudinal axis 532 can be closer to the bridge 506 than the second handling coupling receptacle central longitudinal axis 536. This offset arrangement can be helpful to increase a cross-sectional area at an intersection of the bridge 506 and the first leg 502 and/or the second leg 504 (e.g., amount of material defining the staple body 201 at the intersection). This can help to increase the ability of the staple body 501 to accommodate a load force, applied at the staple 500, in a manner that results in elastic deformation of the staple 500 as the staple 500 moves between a natural, undeformed state and a deformed insertion state upon application/removal of a load force.

The staple 500 can have a thickness 550 that can differ at different regions of the staple 500. For example, the staple 500 can have a bridge thickness 550a at the bridge 506, a leg thickness 550b at the first leg 502 and the second leg 504, and a thickness transition region 555 where the bridge 506 transitions to the respective first leg 502 and the second leg 504. As shown for the illustrated embodiment, the leg thickness 550b can be greater than the bridge thickness 550a (e.g., at a central location of the bridge along the bridge length 516), and the thickness transition region 555 can have a thickness transition region thickness 550c that is greater than the bridge thickness 550a and less than the leg thickness 550b. In particular, the thickness transition region 555 can include an increase in thickness of the staple 500 moving in a direction from the bridge 506 toward the respective leg 502, 504. In one example, the first handling coupling 508 and the first handling coupling receptacle 509 can be located at the thickness transition region 555 adjacent the first leg 502, and the second handling coupling 510 and the second handling coupling receptacle 511 can be located at the thickness transition region 555 adjacent the second leg 504. Such location of the first handling coupling 508 and the first handling coupling receptacle 509 as well as the second handling coupling 510 and the second handling coupling receptacle 511 at the increased thickness portion of the staple 500 can help to increase the strength of the staple 500 for receiving a load force.

As noted, the staple 500 can be configured to have a natural, undeformed state, an example of such state is shown at FIGS. 5A and 5B. The staple 500 can be configured to transition to a deformed insertion state upon application of a load force at the staple 500. The staple 500 can have a biased, compression-inducing state where the first leg 502 and the second leg 504 are angled toward one another, which can help to apply a compression force to urge the bones in which the staple 500 is positioned together, applying a compression force across the separation (e.g., joint, osteotomy, fracture) between the bones such that the end faces of the opposed bones are pressed together.

Upon application of a load force to the staple 500, the staple 500 can be configured to transition from a undeformed state in which the legs of the staple are at their natural or resting positions to a deformed insertion state at which the first and second legs 502, 504 (e.g., end portion 537 of first leg 502 and end portion 538 of second leg 504) are spaced further apart (e.g., and oriented generally parallel to one another) as compared to the natural state. In particular, the staple 500 can be configured such that upon application of the load force at the staple 500, the end portion 537 of first leg 502 is configured to move in a direction 540 (e.g., away from the bridge 506) and the end portion 538 of second leg 504 is configured to move in a direction 542 (e.g., away from the bridge 506) from the undeformed state to the deformed insertion state. Conversely, upon reduction or removal of the applied load force at the staple 500, the staple 500 can be configured such that the end portion 537 of first leg 502 is configured to move in a direction opposite the direction 540 (e.g., toward the bridge 506) from the deformed insertion state back toward the undeformed state, and the end portion 538 of second leg 504 is configured to move in a direction opposite the direction 542 (e.g., toward the bridge 506) from the deformed insertion state back toward the deformed state.

In use, the staple 500 can provide compression across the end faces of the bones into which the staple is inserted. Compression can occur when the legs of the staple are inserted into the bones (e.g., into pre-drilled openings in the bones) at a spacing and/or angle greater than the natural, undeformed configuration of the legs. The staple legs can be deformed to be inserted into the bones and, when the force applied to deform the legs is released, the staple legs can elastically bias toward their unbiased (natural or undeformed) shape. However, the spacing and/or angulation of the legs inserted into the bones can prevent the legs from fully returning to their undeformed state. As a result, the staple can apply a compressive force between the end faces of the bones into which the staple legs are inserted (e.g., with the force directed in the direction of convergence of the staple legs). The compressive force may help promote bone healing and fusion between the bones into which the staple is inserted.

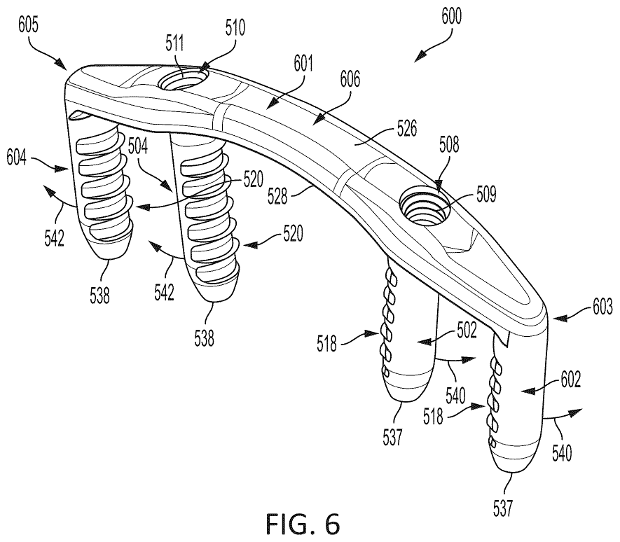

FIG. 6 is a perspective view of another embodiment of a staple 600. The staple 600 can be similar to, or the same as, the staple 500 described previously except as noted here. For example, the staple 600 can include one or more (e.g., each) of the features disclosed herein with respect to the staple 500 (e.g., shape features, dimension features) except as otherwise noted here.

With reference to FIG. 6, the staple 600 can include a body 601 and, in addition to the first leg 502 and the second leg 504 at the body 601, a third leg 602 and a fourth leg 604 at the body 601. For the illustrated embodiment, the staple 600 includes the first leg 502 and the third leg 602 on a first side 603 of a bridge 606. The staple also includes the second leg 504 and the fourth leg 604 on a second side 605 of the bridge 606. As illustrated, the first side 603 is opposite the second side 605 across the bridge 606. The arrangement of the legs 502, 504, 602, 604 of the staple 600 can thus be a four-leg, in-line arrangement.

In some examples, each of the legs 502, 504, 602, 604 can each have an equal length 512, and in the biased compression-inducing state of the staple 600 shown at FIG. 6, the bridge 606 can arch upward away from end portions 537, 538 of legs 502, 504, 602, 604 such that the end portions 537, 538 of legs 602, 604 are at a different elevation than the end portions 537, 538 of legs 502, 504. This arrangement can result the legs 602, 604 beginning insertion into one or more bones before starting insertion of the legs 502, 504 into one or more bones (e.g., because the legs 602, 604 can contact the one or more bones first and the legs 502, 504 contact the one or more bones later after the legs 602, 604 of the staple 600 has begun inserting into the one or more bones).

The bridge 606 can connect the first and third legs 502, 602 to the second and fourth legs 504, 604. The bridge length 516 of the bridge 606 can vary depending on the application and, in some examples, is within a range from 10 mm to 75 mm, such as from 15 mm to 50 mm, from 20 mm to 40 mm, or from 25 mm to 40 mm. In some examples, the bridge length 516 of the bridge 606 can be within a range from 28 mm to 34 mm, which can be sufficient to allow for positioning the bridge across a space (e.g., joint) between bones in the foot while maintaining the legs 502, 602 at one bone and the legs 504, 604 at another bone with the bridge 606 placed across the spaced between such bones. The bridge length 516 for the bridge 606 can be measured from a central longitudinal axis extending through a geometric center of the outermost leg 602 at the first side 603 to a central longitudinal axis extending through a geometric center of the outermost leg 604 at the second side 605.

The staple 600 can have the first handling coupling 508, second handling coupling 510, first handling coupling receptacle 509, and second handling coupling receptacle 511 and one or more (e.g., all) of the features associated therewith as disclosed with respect to the staple 500. Furthermore, the staple 600 can have the material and teeth 518, 520 as well as be configured to transition between the biased compression-inducing state and the deformed insertion state as disclosed with respect to the staple 500.

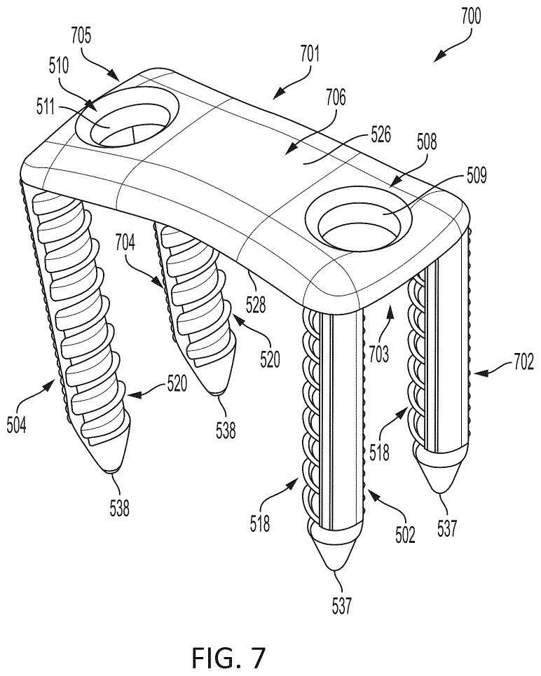

FIG. 7 is a perspective view of yet another embodiment of a staple 700. The staple 700 can be similar to, or the same as, the staple 500 described previously except as noted here. For example, the staple 700 can include one or more (e.g., each) of the features disclosed herein with respect to the staple 500 except as otherwise noted here.

For example, the staple 700 can include a body 701 and, in addition to the first leg 502 and the second leg 504 at the body 701, a third leg 702 and a fourth leg 704 at the body 701. For the illustrated embodiment, the staple 700 includes the first leg 502 and the third leg 702 at a first side 703 of a bridge 706, and thus of the staple 700, and the second leg 504 and the fourth leg 704 at a second side 705 of the bridge 706, and thus of the staple 700. Here the first side 703 is opposite the second side 705. The arrangement of the legs 502, 504, 702, 704 of the staple 700 can thus be a two-by-two leg arrangement, with the legs 502, 504 generally aligned across the bridge 706 and the legs 702, 704 generally aligned across the bridge 706. In some examples, each of the legs 502, 504, 702, 704 can each have an equal length 512, and in the biased compression-inducing state of the staple 700 shown at FIG. 7, the bridge 706 can arch upward away from end portions 537, 538 of legs 502, 504, 702, 704.

The bridge 706 can connect the first and third legs 502, 702 to the second and fourth legs 504, 704. The bridge length 516 of the bridge 706 can range from 15 mm to 20 mm, which can be sufficient to allow for positioning the bridge across a space (e.g., joint) between bones in the foot while maintaining the legs 502, 702 at one bone and legs 504, 704 another bone separated from the one bone by the space. As an example, the bridge length 516 can be as measured from a central longitudinal axis of leg 502 at side 703 of the bridge 706 to a central longitudinal axis of leg 504 at opposite side 705 of the bridge 706 or from a central longitudinal axis of leg 702 at side 703 of the bridge 706 to a central longitudinal axis of leg 704 at opposite side 705 of the bridge 706.

The staple 700 can have the first handling coupling 508, second handling coupling 510, first handling coupling receptacle 509, and second handling coupling receptacle 511 and one or more (e.g., all) of the features associated therewith as disclosed with respect to the staple 500. Furthermore, the staple 700 can have the material and teeth 518, 520 as well as be configured to transition between the biased compression-inducing state and the deformed insertion state as disclosed with respect to the staple 500.

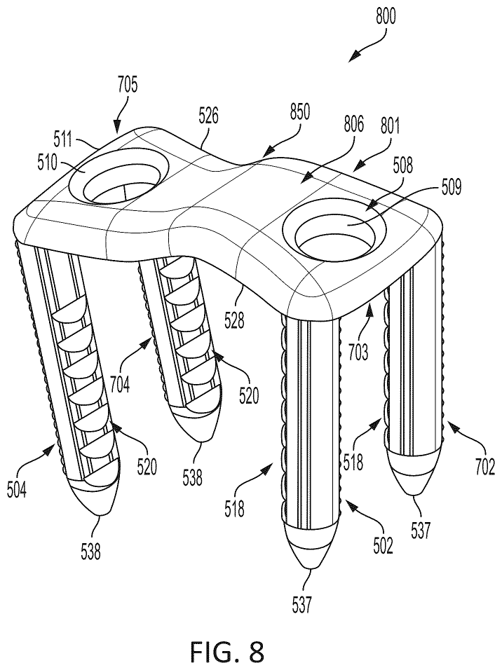

FIG. 8 is a perspective view of an additional embodiment of a staple 800. The staple 800 can be similar to, or the same as, the staple 500 described previously except as noted here. For example, the staple 800 can include one or more (e.g., each) of the features disclosed herein with respect to the staple 500 except as otherwise noted here.

For example, the staple 800 can include a body 801 and, in addition to the first leg 502 and the second leg 504 at the body 801, and the third leg 702 and the fourth leg 704 at the body 801. For the illustrated embodiment, the staple 800 includes the first leg 502 and the third leg 702 at the first side 703 of a bridge 806, and thus of the staple 800, and the second leg 504 and the fourth leg 704 at the second side 705 of the bridge 806, and thus of the staple 800. Here the first side 703 is opposite the second side 705. The arrangement of the legs 502, 504, 702, 704 of the staple 700 can thus be a two-by-two leg arrangement, with the legs 502, 504 generally aligned across the bridge 706 and the legs 702, 704 generally aligned across the bridge 706. In some examples, each of the legs 502, 504, 702, 704 can each have an equal length 512, and in the biased compression-inducing state of the staple 700 shown at FIG. 7, the bridge 806 can arch upward away from end portions 537, 538 of legs 502, 504, 702, 704.

The bridge 806 can connect the first and third legs 502, 702 to the second and fourth legs 504, 704. The bridge length 516 of the bridge 806 can range from 15 mm to 20 mm, which can be sufficient to allow for positioning the bridge across a space (e.g., joint) between bones in the foot while maintaining the legs 502, 702 at one bone and legs 504, 704 another bone separated from the one bone by the space. As an example, the bridge length 516 can be as measured from a central longitudinal axis of leg 502 at side 703 of the bridge 806 to a central longitudinal axis of leg 504 at opposite side 705 of the bridge 806 or from a central longitudinal axis of leg 702 at side 703 of the bridge 806 to a central longitudinal axis of leg 704 at opposite side 705 of the bridge 806.

The staple 800 can further include an elevation transition region 850 at the bridge 806. The elevation transition region 850 can define an elevation change along a length of the bridge 806. For example, as a result of the presence of the elevation transition region 850 at the bridge 806, the side 703 can be at a different elevation than the side 705. For the illustrated embodiment of the staple 800, the side 703 is at a higher elevation than the side 705. The presence of the elevation transition region 850 at the bridge 806 can be useful in facilitating a stable positioning and fixation of the staple 800 at bone surfaces of differing elevations. For example, elevation transition region 850 can be placed at an elevation offset between a metatarsal and opposed cuneiform across the tarsometatarsal joint.

The staple 800 can have the first handling coupling 508, second handling coupling 510, first handling coupling receptacle 509, and second handling coupling receptacle 511 and one or more (e.g., all) of the features associated therewith as disclosed with respect to the staple 500. Furthermore, the staple 700 can have the material and teeth 518, 520 as well as be configured to transition between the biased compression-inducing state and the deformed insertion state as disclosed with respect to the staple 500.

A staple according to the present disclosure (e.g., staple 500, 600, 700, 800) can be fabricated from a variety of different materials. The staple may be fabricated from a biocompatible metal (e.g., titanium, stainless steel, nickel titanium alloy (nitinol)). In one example, the staple is fabricated from titanium (e.g., the staple is formed of a metal consisting of or consisting essential of titanium). The metal forming the staple may be substantially or entirely devoid of nickel. Titanium can be useful in that the metal can resist high energy forces without breakage and can avoid nickel sensitivity issues that may be exhibited by some patients. When so configured, the entire body of the staple (e.g., bridge, legs) can be formed of titanium. During insertion, the legs of the titanium staple may be elastically deformed, allowing the legs to return to toward their original, undeformed position. Other materials, including combinations of different materials, may be used in other configurations of a staple according to the disclosure.

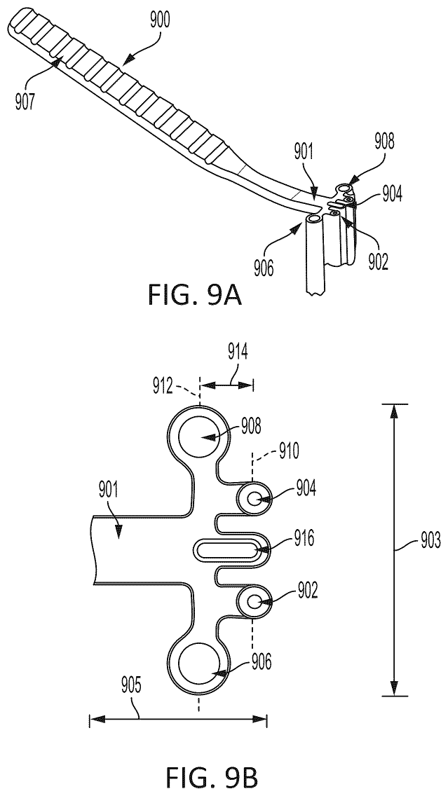

FIGS. 9A and 9B illustrate an embodiment of a guide 900. FIG. 9A is perspective view of the guide 900, and FIG. 9B is a top plan view of a body 901 of the guide 900. The guide 900 can be useful in helping to prepare and facilitate desired placement of an implant, such as any of the staple embodiments disclosed herein, at and across target bones for fixation and fusion.

The guide 900 can include a body 901 and a handle 907. The body 901 can define a body length 903 and a body width 905, with the body width 905 being transverse to the body length 903. The handle 907 can be connected to the body 901 and extend away from the body 901. The handle 907 can be configured to be held in a hand of a user and, in the illustrated embodiments, the handle 907 angles upward away from the body 901 as the handle 907 extends out from the body 901 so as to create a more ergonomically adapted handle 907 orientation that also generally facilitates convenient placement of the body 901 in contact with one or more bones (or tissue overlaying such one or more bones).

The guide 900 can include a first wire aperture 902 extending through the body 901 and a second wire aperture 904 at the body 901. The first wire aperture 902 can be configured to receive a first wire therethrough, and the second wire aperture 904 can be configured to receive a second wire therethrough. The first and second wire apertures 902, 904 can help to facilitate desired placement of such wires in relatively small target bones by providing an alignment guide that places wires extending through the apertures at consistent, repeatable positioning and/or spacing. The first and second wire apertures 902, 904 can be spaced apart along the body length 903 of the body 901 a distance that corresponds to a space between two relatively small bones in the foot (e.g., spaced apart a distance that corresponds to the TMT joint space separating the medial cuneiform and the first metatarsal).

As one example, the guide 900 can be configured, at least in part, for use as a drill guide. In such an example, the guide 900 can further include a first drill guide aperture 906 extending through the body 901 and a second drill guide aperture 908 extending through the body 901. The first drill guide aperture 906 and the second drill guide aperture 908 can be configured to receive a drill or other tool capable of creating an implant hole in underlying bone therethrough. Similar to the first and second wire apertures 902, 904, the first and second drill guide apertures 906, 908 can be spaced apart along the body length 903 of the body 901 a distance that corresponds to a space between two relatively small bones in the foot (e.g., spaced apart a distance that corresponds to the TMT joint space separating the medial cuneiform and the first metatarsal).

For the illustrated embodiment of the guide 900, the body 901 includes the first and second wire apertures 902, 904 generally aligned on a first common axis 910 that runs parallel to the body length 903, and the body 901 includes the first and second drill guide apertures 906, 908 generally aligned on a second common axis 912 that runs parallel to the body length 903, with the first common axis 910 being offset from the second common axis 912 a distance 914 in a direction parallel to the body width 905. In the illustrated embodiment of the guide 900, the first wire aperture 902 and the second wire aperture 904 are positioned closer together along the body length 903 than the first drill guide 906 and the second drill guide 908. In this illustrated embodiment, the first drill guide aperture 906 and the second drill guide aperture 908 are positioned at opposite end portions of the body length 903 from one another.

The guide 900 can further include a seeker receiving aperture 916 at the body 901. The seeker receiving aperture 916 can be configured to receive a seeker. The seeker receiving aperture 916 can be located at the body 901 at a location on the body 901 generally corresponding to a location of a space between bones when the guide 900 is placed at the target anatomy. As shown here, the seeker receiving aperture 916 can be positioned at the body 901 between the first wire aperture 902 and the second wire aperture 904 along the body length 903 and between the first drill guide 906 and the second drill guide 908 along the body length 903. As such, with the first wire aperture 902 and the first drill guide 906 located at the body 901 at a location corresponding to a first bone, when the body 901 is placed at the target anatomy, and the second wire aperture 904 and the second drill guide 908 located at the body 901 at a location corresponding to a second bone, when the body 901 is placed at the target anatomy spaced apart from the first bone by a space, the seeker receiving aperture 916 can be located at the body 901 between these features at a location on the body 901 generally corresponding to a location of this space between these bones when the guide 900 is placed at the target anatomy. In one example, in addition to or as an alternative to the seeker receiving aperture 916, the guide 900 can include a seeker integral to the body 901 at the location of the seeker receiving aperture 916, and this integral seeker can extend out from the body 901 in a direction of the space between bones when the guide 900 is placed at the target anatomy. In another example, a seeker can be separate from the body 901 and inserted into (e.g., and through) the seeker receiving aperture 916 as is illustrated at subsequent figures.

Features describes as wires herein can be implemented using a k-wire, Steinman pin, and/or other surgically acceptable wire. Each wire may or may not have a threaded distal end and/or sharpened distal tip to facilitate insertion into bone. Each wire may have a circular cross-sectional shape or other polygonal (e.g., square, triangular, hexagonal) or arcuate shape.

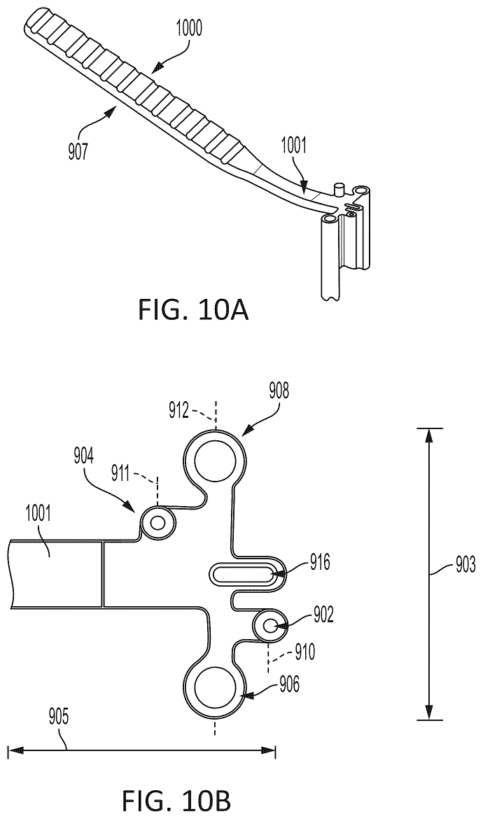

FIGS. 10A and 10B illustrate an embodiment of a guide 1000. FIG. 10A is perspective view of the guide 1000, and FIG. 10B is a top plan view of a body 1001 of the guide 1000. The guide 1000 can be similar to, or the same as, guide 900 described previously except as noted here. For example, the guide 1000 can include one or more (e.g., each) of the features disclosed herein with respect to the guide 900 except as otherwise noted here.

For example, the guide 1000 can have an offset arrangement of wire apertures 902, 904. The body 1001 can have the body length 903 and the body width 905, and the first wire aperture 902 and the second wire aperture 904 can be located opposite one another along the body length 903 and opposite one another along the body width 905. The body 1001 can include the first and second drill guide apertures 906, 908 generally aligned on the second common axis 912 that runs parallel to the body length 903, and the body 1001 can include the first wire aperture 902 at one side of the second common axis 912 and the second wire aperture 904 at another, opposite side of the second common axis 912 such that the first and second wire apertures 902, 904 can be spaced apart from one other in a direction parallel to the body width 905. While offset in the direction parallel to the body width 905, the first and second wire apertures 902, 904 can be located closer together as measured in a direction parallel to the body length 903 than the first and second drill guide apertures 906, 908 are in the direction parallel to the body length 903. This offset arrangement of the first and second wire apertures 902, 904 can be useful in facilitating inverse (e.g., mirror-image) orientation placement of the guide 1000 at the target anatomy and thereby can help to reduce instances necessitating wire relocation at a bone.

A further embodiment of a guide can be similar to, or the same as, the guide 900 or the guide 1000 except that such additional guide (e.g., drill guide) embodiment can have more than two wire apertures at the body. In example of such a further guide embodiment, the body of the guide can include at least four wire apertures. For instance, two such wire apertures could be located at one side portion of the body (e.g., a first side portion at or near a first end portion along the body width at one side of the drill guide apertures) and two other such wire apertures could be located an another, opposite side portion of the body (e.g., a second side portion at or near a second, opposite end portion along the body width at another, opposite side of the drill guide apertures). When using such a further embodiment of a guide, a user can choose a side portion of the body (e.g., a side portion positioned nearest a medial side of the foot) and insert one or more wires through the at least two wire apertures located at that chosen side of the body.

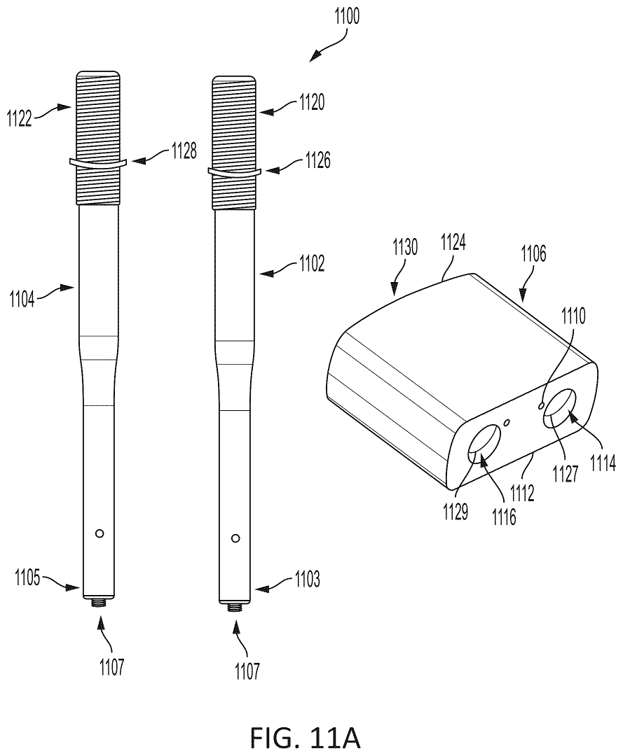

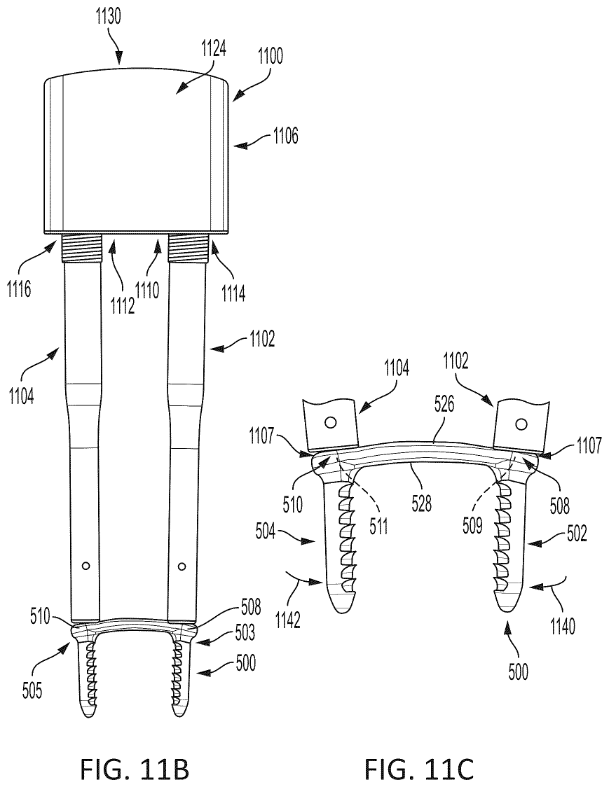

FIGS. 11A-11C illustrate an embodiment of an inserter 1100. FIG. 11A shows disassembled components of the inserter 1100, FIG. 11B shows assembled components of the inserter 1100, and FIG. 11C shows the inserter 1100 operatively connected to staple 500.

The inserter 1100 can include a first coupling shaft 1102, a second coupling shaft 1104, and a connector 1106. The first and second coupling shafts 1102, 1104 can be configured to operatively connect to an implant, such as a staple. The illustrated embodiment of the inserter 1100 shows the first and second coupling shafts 1102, 1104 each configured to operatively couple to the staple 500 (or another staple configuration as described herein). The connector 1106 can be configured to join the first coupling shaft 1102 and the second coupling shaft 1104, for instance as shown at the example of FIG. 11B.

In particular, the first coupling shaft 1102 can be configured to operatively couple to the staple 500 at the first handling coupling 508, and the second coupling shaft 1104 can be configured to operatively couple to the second handling coupling 510. For example, the first coupling shaft 1102 can have a distal end portion 1103 and the second coupling shaft 1104 can have a distal end portion 1105, and each of such distal end portions 1103, 1105 can include an implant coupling member 1107. The coupling member 1107 at the distal end portion 1103 of the first coupling shaft 1102 can be configured to operatively connect to a complementary coupling member at the first handling coupling 508, and the coupling member 1107 at the distal end portion 1105 of the second coupling shaft 1104 can be configured to operatively connect to a complementary coupling member at the second handling coupling 510. In this way, the inserter 1100 can include the first coupling shaft 1102 connected to a first side of the implant, such as the first side 503 of the staple 500, and the second coupling shaft 1104 connected to a second side of the implant, such as the second side 505 of the staple 500.

As one such specific example, each of the first and second handling couplings 508, 510 can include threading as a type of complementary coupling member thereat. The coupling member 1107 at the distal end portions 1103, 1105 of the respective first and second coupling shafts 1102, 1104 can include threading that is configured to operatively couple to the complementary threading at the respective first and second handling couplings 508, 510. Thus, in this particular example, operatively coupling the inserter 1100 to an implant, such as the staple 500, can include threadingly inserting the first coupling shaft 1102 into the first handling coupling 508 (e.g., from the top surface 526 of the staple 500) and threadingly inserting the second coupling shaft 1104 into the second handling coupling 510 (e.g., from the top surface 526 of the staple 500). Other types of mechanical connections than threading can be used.

As best seen at FIG. 11C, each of the first coupling shaft 1102 and the second coupling shaft 1104 can be configured to couple to the respective first and second handling coupling receptacles 509, 511 at a location between the top surface 526 of the staple 500 and the bottom surface 528 of the staple 500. For example, the first coupling shaft 1102 can operatively couple to the first handling coupling 508 from the top surface 526 and in a direction toward the bottom surface 528 but without the first coupling shaft 1102 extending out from the bottom surface 528. Likewise, the second coupling shaft 1104 can operatively couple to the second handling coupling 510 from the top surface 526 and in a direction toward the bottom surface 528 but without the second coupling shaft 1104 extending out from the bottom surface 528. As one such example, the coupling member 1107 at the first coupling shaft 1102 can extend within the first handling coupling receptacle 509 such that a distal end of the coupling member 1107 at the first coupling shaft 1102 is contained within the staple 500 (e.g., within the first handling coupling receptacle 509). Similarly, the coupling member 1107 at the second coupling shaft 1104 can extend within the second handling coupling receptacle 511 such that a distal end of the coupling member 1107 at the second coupling shaft 1104 is contained within the staple 500 (e.g., within the second handling coupling receptacle 511).

The inserter 1100 can be configured to couple to the staple through the top surface 526 of the staple 500. The first handling coupling 508 can extend through the top surface 526 of the staple 500 on the first side 503 of the staple 500, and the second handling coupling 510 can extend through the top surface 526 of the staple 500 on the second side 505 of the staple 500. The inserter 1100 can be configured to couple to the first handling coupling 508 through the top surface 526 of the staple 500 without extending under the bottom surface 528 of the staple 500, and the inserter 1100 can be configured to couple to the second handling coupling 510 through the top surface 526 of the staple 500 without extending under the bottom surface 528 of the staple 500. In one exemplary application where the staple 500 is to be placed at a metatarsal, at a cuneiform, and bridging across a space (e.g., joint space, such as the TMT joint space) between the metatarsal and cuneiform, when the inserter 1100 is connected to the first handling coupling 508 and/or the second handling coupling 510 through the top surface 526 without extending under the bottom surface 528 of the staple 500, the bottom surface 528 of the staple 500 can be configured to directly contact at least one of a metatarsal and a cuneiform without any inserter 1100 structure present between the bottom surface 528 and the at least one of the metatarsal and the cuneiform. This configuration can allow the bottom surface 528 of the staple 500 to be more flushly placed at the metatarsal and/or cuneiform as compared to a configuration where an inserter structure is present between the bottom surface 528 and the metatarsal and/or cuneiform when such inserter is coupled to the staple 500. With the inserter 1100 coupled to the first and second handling couplings 508, 510 through the top surface 526 of the staple 500 without extending under the bottom surface 528 of the staple 500, the inserter 1100 can be configured to apply the load force at the staple 500 to cause the first second legs 502, 504 to move apart from one another.

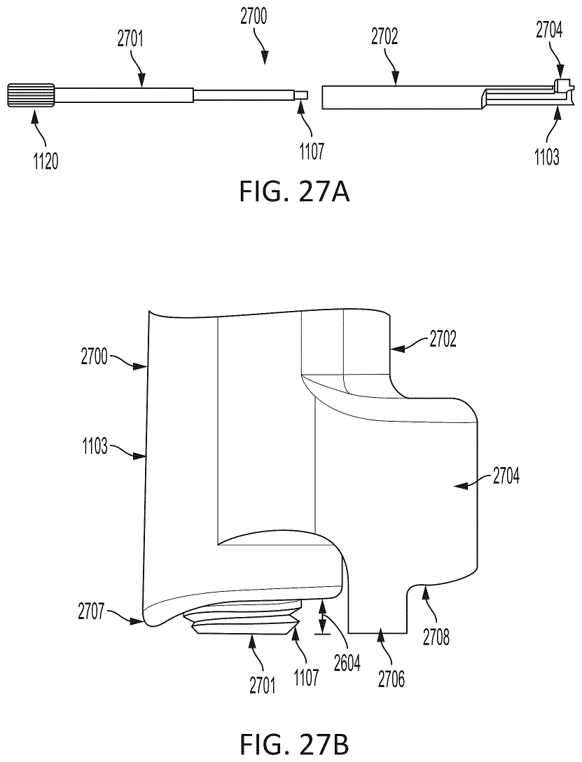

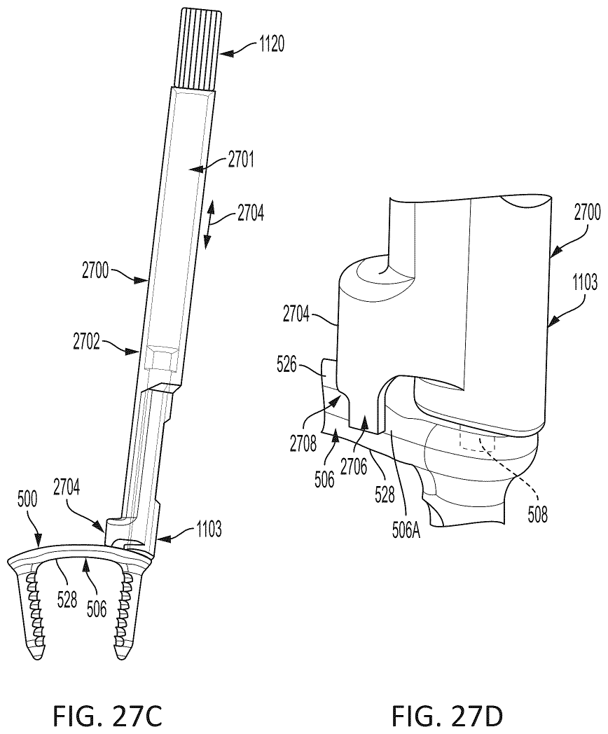

In some additional or alternative examples where the inserter 1100 is configured to couple to the staple 500 through the top surface 526 of the staple 500, the inserter 1100 can be configured to connect to the first side 503 of the staple 500 through the top surface 526 without extending under the bottom surface 528 and without contacting an outer perimeter of the first side 503 of the staple 500, and the inserter 1100 can be configured to connect to the second side 505 of the staple 500 through the top surface 526 without extending under the bottom surface 528 and without contacting an outer perimeter of the second side 505 of the staple 500. For example, the inserter 1100 can be configured to connect to the first side 503 of the staple 500 through the top surface 526 without extending under the bottom surface 528 and without contacting an outer perimeter of the first side 503 of the staple 500 formed by the sidewall 506A (reference character shown at FIG. 27D) which connects the top surface 526 of the staple 500 to the bottom surface 528 of the staple 500, and, similarly, the inserter 1100 can be configured to connect to the second side 505 of the staple 500 through the top surface 526 without extending under the bottom surface 528 and without contacting an outer perimeter of the second side 505 of the staple 500 formed by the sidewall 506A.

In some additional or alternative examples where the inserter 1100 is configured to couple to the staple 500 through the top surface 526 of the staple 500, the inserter 1100 can be configured to connect to the first side 503 of the staple 500 through the top surface 526 without extending under the bottom surface 528 such that, when the inserter 1100 is connected to the first handling coupling 508, the bottom surface 528 of the staple 500 is configured to directly contact the one or more bones without any inserter 1100 structure between the bottom surface 528 and such one or more bones. Similarly, the inserter 1100 can further be configured to connect to the second side 505 of the staple 500 through the top surface 526 without extending under the bottom surface 528 such that, when the inserter 1100 is connected to the second handling coupling 510, the bottom surface 528 of the staple 500 is configured to directly contact the one or more bones without any inserter 1100 structure between the bottom surface 528 and such one or more bones.

In some additional or alternative examples where the inserter 1100 is configured to couple to the staple 500 through the top surface 526 of the staple 500, the staple 500 and the inserter 1100 can be configured such that, when the inserter 1100 is coupled to the first handling coupling 508, the inserter 1100 contacts first handling coupling 508 and the inserter 1100 is isolated at the first handling coupling 508 from any contact with a perimeter sidewall (e.g., sidewall 506A) of the staple 500 connecting the top surface 526 of the staple 500 to the bottom surface 528 of the staple 500. Similarly, the staple 500 and inserter 1100 can further be configured such that, when the inserter 1100 is coupled to the second handling coupling 510, the inserter 1100 contacts the second handling coupling 510 and the inserter 1100 is isolated at the second handling coupling 510 from any contact with the perimeter sidewall (e.g., sidewall 506A) of the staple 500. As one particular such example, the staple 500 and the inserter 1100 can be configured such that, when the inserter 1100 is coupled to the staple 500 at the first handling coupling 508, the inserter 1100 is confined to contacting the staple 500 at only the first handling coupling receptacle 509 defined by the first handling coupling 508, and, when the inserter 1100 is coupled to the staple 500 at the second handling coupling 510, the inserter 1100 is confined to contacting the staple 500 at only the second handling coupling receptacle 511 defined by the second handling coupling 510.

In various embodiments, the inserter 11100 (e.g., coupling shafts 1102, 1104) can be configured to couple to the staple 500 through the top surface 526 of the staple 500 such that only the staple 500, and no portion of the inserter 1100 (e.g., no portion of the coupling shafts 1102, 1104), defines a contact interface with the one or more bones at which the staple 500 is being inserted into. This coupled configuration of the staple 500 and inserter 1100 (e.g., coupling shafts 1102, 1104) that results in staple only contact at the one or more bones at which the staple 500 is being inserted into can help to insert the staple 500 flushly at the one or more bones because no inserter 1100 structure (e.g., no coupling shaft 1102, 1104 structure) is present at a location to contact the one or more bones which would prevent flush insertion of the staple 500 at the one or more bones. As one example, the inserter 1100 (e.g., coupling shafts 1102, 1104) can be configured to couple to the staple 500 by contacting the staple 500 at only locations above the bottom surface 528 of the staple 500. For the illustrated embodiment of the coupling shafts 1102, 1104, the coupling shafts 1102, 1104 can be configured to contact the staple 500 at only locations above the bottom surface 528 of the staple 500. As shown at this illustrated embodiment at FIGS. 11B and 11C, when the coupling shafts 1102, 1104 are coupled to the staple 500, the implant coupling member 1107 of each coupling shaft 1102, 1104 contacts the staple 500 at only staple portions above the bottom surface 528 of the staple 500. In such embodiment, when the inserter 1100 is coupled to the staple 500, there can be no inserter 1100 structure (i) in contact with the outer perimeter sidewall 506a of the staple 500 and (ii) below the bottom surface 528 of the staple 500.

In a further embodiment, to help provide added stability when applying a load force at the implant (e.g., staple 500), the first and/or second coupling shaft 1102, 1104 can include a shaft stabilizing arm. When so included, the shaft stabilizing arm can be included at the respective distal end portion 1103, 1105 of the respective coupling shaft 1102, 1104 and extend in a direction parallel to a central longitudinal axis of the respective coupling shaft 1102, 110. Where the implant is a staple, the shaft coupling arm can be configured to contact the bridge of the staple (e.g., at a top and/or side surface of the bridge but not a bottom surface of the bridge) when the coupling member 1107 of the respective shaft 1102, 1104 is at the respective handling coupling at the staple. The inclusion of such shaft stabilizing arm can be useful to help provide additional stability during the application of a load force at the implant (e.g., staple) and placement of the implant (e.g., staple) at the target anatomy.

In some examples where the inserter 1100 is configured to couple to the staple 500 through the top surface 526 of the staple 500 and includes one or more shaft stabilizing arm(s), the inserter 1100 can further be configured to couple to the first handling coupling 508 through the top surface 526 of the staple 500 without extending down an entire thickness 550 of a perimeter sidewall (e.g., sidewall 506A) of the staple 500 connecting the top surface 526 of the staple 500 to the bottom surface 528 of the staple 500. Similarly, the inserter 1100 can be further configured to couple to the second handling coupling 510 through the top surface 526 of the staple 500 without extending down the entire thickness 550 of the perimeter sidewall (e.g., sidewall 506A) of the staple 500 connecting the top surface 526 of the staple 500 to the bottom surface 528 of the staple 500. In an alternate example where the inserter 1100 is configured to couple to the staple 500 through the top surface 526 of the staple 500, the inserter 1100 can further be configured to: (i) couple to the first handling coupling 508 through the top surface 526 of the staple 500 without extending down more than three-quarters of the perimeter sidewall (e.g., sidewall 506A) of the staple 500 connecting the top surface 526 of the staple 500 to the bottom surface 528 of the staple 500, and (ii) couple to the second handling coupling 510 through the top surface 526 of the staple 500 without extending down more than three-quarters of the perimeter sidewall (e.g., sidewall 506A) of the staple 500 connecting the top surface 526 of the staple 500 to the bottom surface 528 of the staple 500. In another alternate example where the inserter 1100 is configured to couple to the staple 500 through the top surface 526 of the staple 500, the inserter 1100 can further be configured to: (i) couple to the first handling coupling 508 through the top surface 526 of the staple 500 without extending down more than half of the perimeter sidewall (e.g., sidewall 506A) of the staple 500 connecting the top surface 526 of the staple 500 to the bottom surface 528 of the staple 500, and (ii) couple to the second handling coupling 510 through the top surface 526 of the staple 500 without extending down more than half of the perimeter sidewall (e.g., sidewall 506A) of the staple 500 connecting the top surface 526 of the staple 500 to the bottom surface 528 of the staple 500. In these noted examples, the thickness 550 of the perimeter sidewall (e.g., sidewall 506A) of the staple 500 connecting the top surface 526 of the staple 500 to the bottom surface 528 of the staple 500 can be at least 0.5 mm. As such, in the example where the inserter 1100 is configured to couple to the first and second handling couplings 508, 510 without extending down more than three-quarters of the perimeter sidewall (e.g., sidewall 506A) of the staple 500, the inserter can be configured to so couple without extending down more than three-quarters of the of the at least 0.5 mm thickness 550 at the perimeter sidewall (e.g., sidewall 506A) of the staple 500. And in the example where the inserter 1100 is configured to couple to the first and second handling couplings 508, 510 without extending down more than half of the perimeter sidewall (e.g., sidewall 506A) of the staple 500, the inserter can be configured to so couple without extending down more than half of the of the at least 0.5 mm thickness 550 at the perimeter sidewall (e.g., sidewall 506A) of the staple 500.

The inserter 1100 can further include a first wire receiving opening 1110, a second wire receiving opening 1112, a first receptacle 1114, and a second receptacle 1116. The first wire receiving opening 1110 can be configured to receive a first wire, and the second wire receiving opening 1112 can be configured to receive a second wire. The first receptacle 1114 can be configured to receive and hold the first coupling shaft 1102, for instance a proximal end portion 1120 of the first coupling shaft 1102, and the second receptacle 1116 can be configured to receive and hold the second coupling shaft 1104, for instance a proximal end portion 1122 of the second coupling shaft 1104. For the illustrated embodiment, each of the first wire receiving opening 1110, the second wire receiving opening 1112, the first receptacle 1114, and the second receptacle 1116 is included at the connector 1106.

As noted, the connector 1106 can be configured to join the first coupling shaft 1102 and the second coupling shaft 1104, for instance as shown at the example of FIG. 11B. As one example, the connector 1106 can be configured to join the first and second coupling shafts 1102, 1104 by receiving the first coupling shaft 1102 at the first receptacle 1114 at the connector 1106 and receiving the second coupling shaft 1104 at the second receptacle 1116 at the connector 1106. As such, with the receptacles 1114, 1116 at the connector 1106 receiving and holding the respective coupling shaft 1102, 1104, the connector 1106 can removably join the coupling shafts 1102, 1104.

To help facilitate a more robust joining of the coupling shafts 1102, 1104 via the connector 1106, the first coupling shaft 1102 can include a first retention feature 1126 and the second coupling shaft 1104 can include a second retention feature 1128. For example, the first retention feature 1126 can be located at or near the proximal end portion 1120 of the first coupling shaft 1102, and the second retention feature 1128 can be located at or near the proximal end portion 1122 of the second coupling shaft 1104. The first retention feature 126 can be configured to help hold the first coupling shaft 1102 in the first receptacle 1114 and the second retention feature 1128 can be configured to help hold the second coupling shaft 1104 in the second receptacle 1116. In one such further example, the connector 1106 can include a first retention mating feature 1127 at the first receptacle 1114 and a second retention mating feature 1129 at the second receptacle 1116. The first retention mating feature 1127 can be complementary to the first retention feature 1126 and configured to receive and hold the first retention feature 1126, and the second retention mating feature 1129 can be complementary to the second retention feature 1128 and configured to receive and hold the second retention feature 1128. The first retention feature 1126 and the first retention mating feature 1127 as well as the second retention feature 1128 and the second retention mating feature 1129 can take any of a variety of suitable forms of complementary connector pairs, such as, for one suitable, non-limiting example, complementary structures that create an interference fit. As one such specific example, the first retention feature 1126 and the first retention mating feature 1127 as well as the second retention feature 1128 and the second retention mating feature 1129 can be complementary connector pairs that provide a retention force in a direction generally parallel to a longitudinal axis of the coupling shafts 1102, 1104 and configured to release this retention force upon movement of at least one of the complementary connectors of the pair (e.g., movement of one of the coupling shaft 1102 and the receptacle 1114 and movement of one of the coupling shaft 1104 and the receptacle 1116) in a direction generally transverse to longitudinal axis of the coupling shafts 1102, 1104.

As one specific such example, the connector 1106 as shown for the illustrated embodiment of the inserter 1100 can include a cap 1124. For the illustrated embodiment of the inserter 1100, the cap 1124 can be configured to be positioned over the proximal end portion 1120 (e.g., opposite the implant, such as the staple 500) of the first coupling shaft 1102 and over the proximal end portion 1122 (.g., opposite the implant, such as the staple 500) of the second coupling shaft 1104. For instance, the cap 1124 can be configured to join the first and second coupling shafts 1102, 1104 by placing the first receptacle 1114 at the cap 1124 over the proximal end portion 1120 of the first coupling shaft 1102 and the second receptacle 1116 at the cap 1124 over the proximal end portion 1122 of the second coupling shaft 1104, and then moving the cap 1124 as so positioned relative to the first and second coupling shafts 1102, 1104 (e.g., in a direction toward one or more bones) such that the proximal end portions 1120, 1122 are received and held at the respective receptacles 1114, 1116. Likewise, the cap 1124 can similarly be configured to receive and hold first and second wires, positioned at one or more bones, at respective first and second wire receiving openings 1110, 1112 as the cap 1124 is moved relative to such wires (e.g., in a direction toward one or more bones).

The cap 1124 can further include a surface contour 1130. The surface contour 1130 can be adapted to fit at a hand of a user. For the illustrated example, the surface contour 1130 can have a highest elevation at a location between the receptacles 1114, 1116 and a lowest elevation outside of the receptacles 1114, 1116 such that the surface contour 1130 angles downward toward the implant, such as the staple 500, when moving along the surface contour 1130 away from the highest elevation between the receptacles 1114, 1116. Accordingly, when positioning the implant, such as the staple 500, in contact with the first bone and the second bone, a user's hand can tamp at the surface contour 1130 of the cap 1124 to apply insertion force at the implant, such as the staple 500.

The inserter 1100 can be configured to place the implant, such as the staple 500, in one or more bones. For example, the inserter 1100 can be operatively connected to the staple 500 via the first and second coupling shafts 1102, 1104, and the connector 1106 (e.g., cap 1124) can be joined to the first and second coupling shafts 1102, 1104, such as shown at the example of FIG. 11B. When the connector 1106 is joined to the first coupling shaft 1102 and the second coupling shaft 1104, such as shown at the example of FIG. 11B, the connector 1106 can be configured to bias the first coupling shaft 1102 and the second coupling shaft 1104 toward each other to apply a load force to the staple 500. When the first coupling shaft 1102 and the second coupling shaft 1104 are so biased toward each other to apply the load force to the staple 500, the first leg 502 and the second leg 504 can be oriented generally parallel to one another, such as shown at the example of FIG. 11B. This application of the load force to the staple 500 can cause the legs 502, 504 to move away from each other to the generally parallel orientation which can be a configuration useful for inserting the staple 500 in and/or across the bones. Then, after the staple 500 has been inserted as desired at and across the bones, the connector 1106 can be removed from at least one of the first coupling shaft 1102 and the second coupling shaft 1104 to cause the first leg 502 and the second leg 504 to move toward one another. Specifically, removing the connector 1106 can cause the first leg 502 to move in a direction 1140 toward the second leg 504 and cause the second leg 504 to move in a direction 1142 toward the first leg 502.

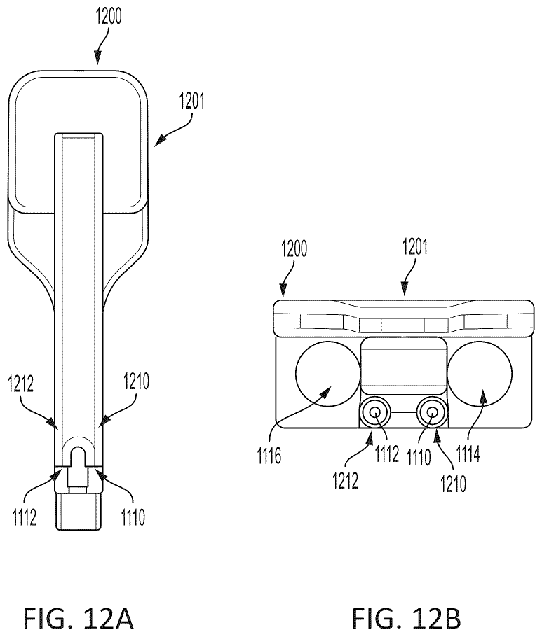

FIGS. 12A and 12B illustrate another embodiment of an inserter 1200. FIG. 12A shows a side elevational view of a longitudinal cross-section of the inserter 1200, and FIG. 12B shows a bottom plan view of a cap 1201 of the inserter 1200. The inserter 1200 can be similar to, or the same as, inserter 1100 described previously except as noted here. For example, the inserter 1200 can include one or more (e.g., each) of the features disclosed herein with respect to the inserter 1100 except as otherwise noted here.

The inserter 1200 can include the first and second coupling shafts 1102, 1104, such as shown for the inserter 1100. Likewise, the inserter 1200 can include the first wire receiving opening 1110, the second wire receiving opening 1112, the first receptacle 1114, and the second receptacle 1116. As compared to the inserter 1100, the inserter 1200 can additionally include a first wire receiving sleeve 1210 extending from the first wire receiving opening 1110 and a second wire receiving sleeve 1212 extending from the second wire receiving opening 1112. The wire receiving sleeves 1210, 1212 can be elongated in a direction parallel to a longitudinal axis of the inserter 1200 and help to provide additional stability and retention at the interface of the cap 1201 and the first and second wires received at the cap 1201.

As shown for the illustrated embodiment of the inserter 1200, the first and second wire receiving openings 1110, 1112 can be included in an aligned arrangement. For instance, the first and second wire receiving openings 1110, 1112 can have an aligned arrangement and spacing therebetween that corresponds (e.g., matches) that of the first wire aperture 902 and the second wire aperture 904 of the guide 900. For instance, the first and second wire receiving openings 1110, 1112 can be at a common side of the cap 1201 and each located between the receptacles 1114, 1116. Accordingly, the first and second wire receiving openings 1110, 1112 at the inserter 1200 can be configured to receive therein first and second wires placed using the respective first and second wire apertures 902, 904 of the guide 900.

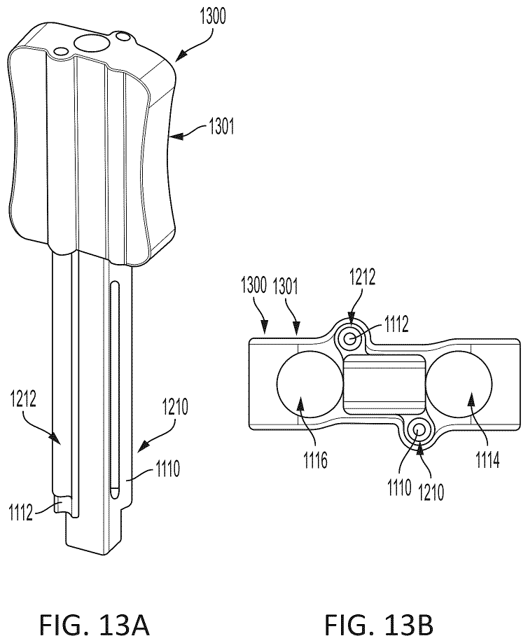

FIGS. 13A and 13B illustrate yet another embodiment of an inserter 1300. FIG. 13A shows a perspective view of the inserter 1300, and FIG. 13B shows a bottom plan view of a cap 1301 of the inserter 1300. The inserter 1300 can be similar to, or the same as, inserter 1200 described previously except as noted here. For example, the inserter 1300 can include one or more (e.g., each) of the features disclosed herein with respect to the inserter 1200 except as otherwise noted here.

The inserter 1300 can include the first and second coupling shafts 1102, 1104, such as shown for the inserter 1100, the first wire receiving opening 1110, the second wire receiving opening 1112, the first receptacle 1114, the second receptacle 1116, the first wire receiving sleeve 1210 extending from the first wire receiving opening 1110, and the second wire receiving sleeve 1212 extending from the second wire receiving opening 1112.

As shown for the illustrated embodiment of the inserter 1300, the first and second wire receiving openings 1110, 1112 can be included in an offset arrangement. For instance, the first and second wire receiving openings 1110, 1112 can have an offset arrangement and spacing therebetween that corresponds (e.g., matches) that of the first wire aperture 902 and the second wire aperture 904 of the guide 1000. For instance, the first and second wire receiving openings 1110, 1112 can be at opposite sides of the cap 1301 and each located between the receptacles 1114, 1116. Accordingly, the first and second wire receiving openings 1110, 1112 at the inserter 1300 can be configured to receive therein first and second wires placed using the respective first and second wire apertures 902, 904 of the guide 1000.

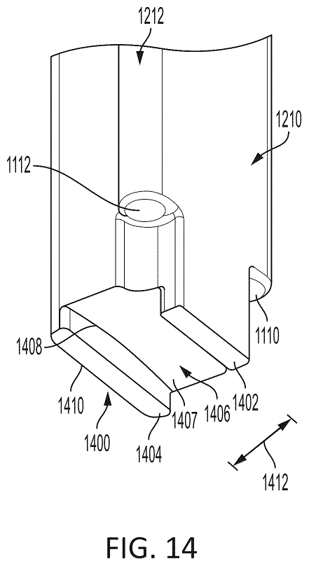

FIG. 14 is a perspective view of an implant tamp feature 1400 that can be included, for instance, at any one or more of the inserter embodiments 1100, 1200, 1300. The implant tamp feature 1400 can be included, for example, at or near a distal end, which is configured to contact and operatively connect to an implant, such as the staple 500, of any one or more of the inserter embodiments 1100, 1200, 1300.

The implant tamp feature 1400 can include a first wall 1402, a second wall 1404, and an implant receiving slot 1406 defined between the first and second walls 1402, 1404. As illustrated for the embodiment shown here, the implant receiving slot 1406 can be defined by a base surface 1407 that extends a distance 1412 from the first wall 1402 to the second wall 1404. Each of the first and second walls 1402, 1404 can extend out from the base surface 1407 such that a proximal end 1408 of each wall 1402, 1404 can be at the base surface 1407 and a distal end 1410 of each wall 1402, 1404 can be spaced outward and apart from the base surface 1407. The implant receiving slot 1406 can be sized to receive thereat between the walls 140, 1404 an implant. For example, where the implant is the staple 500, the implant receiving slot 1406 can be sized to receive therein the staple 500. In this example, the distance 1412 between the walls 1402, 1404, and thus defining a width of the implant receiving slot 1406, can be slightly larger than the bridge width 570 of the bridge 506 of the staple 500.

The implant tamp feature 1400 can be configured to transfer a tamping force applied by a user's hand at the inserter to the implant received at the implant receiving slot 1406. By confining the implant, such as the staple 500, to the distance 1412 at the implant receiving slot 1406 between the walls 1402, 1404, a greater degree of tamping force can be transferred from the inserter to the implant, such as the staple 500. Thus, the implant tamping feature 1400 can help to increase efficiency in inserting the implant, such as the staple 500, at bone(s).

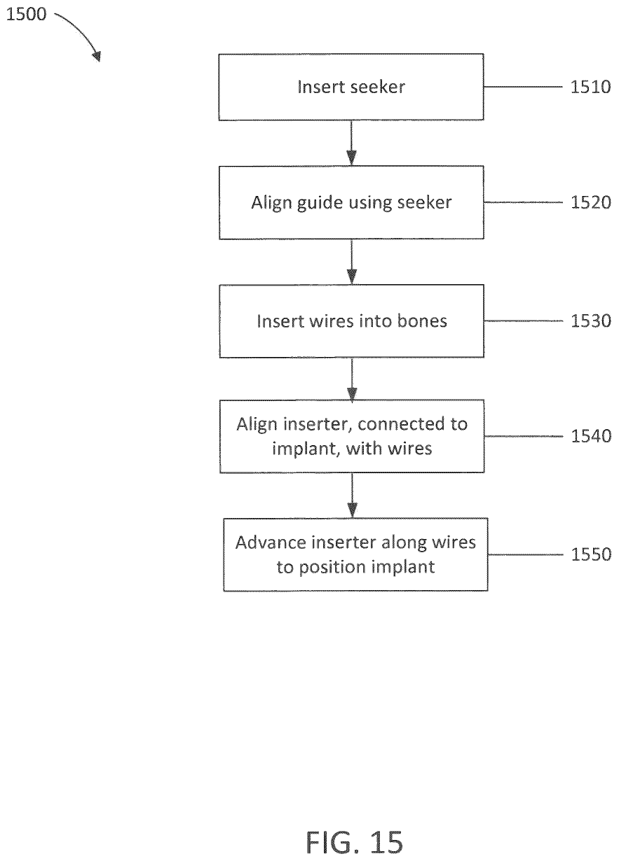

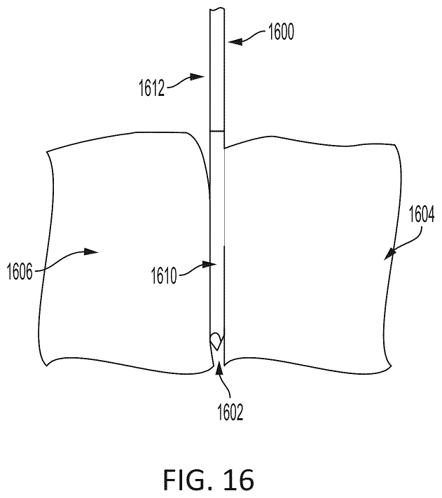

FIG. 15 is a flow diagram of an embodiment of a method 1500. The method 1500 can be used as, for example, a surgical technique for positioning an implant, such as a staple, to facilitate fixating bones for fusion. FIGS. 16-21 will be referenced in conjunction with the method 1500 to provide exemplary illustrations of non-limiting embodiments of steps of the method 1500.

At step 1510, the method 1500 can include inserting a seeker. For example, step 1510 can include inserting a seeker into a space (e.g., a joint) separating a first bone from a second bone. As one example, the first bone can be a metatarsal (e.g., a first metatarsal), the second bone can be a cuneiform (e.g., a medial cuneiform), and the space separating the first bone from the second bone can be a tarsometatarsal joint space separating the metatarsal from the cuneiform.

FIG. 16 illustrates a side elevational view of an embodiment of a seeker 1600 that is inserted into a space 1602 (e.g., a joint space, such as a TMT joint space) separating a first bone 1604 (e.g., a metatarsal, such as a first metatarsal) and a second bone 1606 (e.g., a cuneiform, such as a medial cuneiform). As shows here, the seeker 1600 can include a first portion 1610 and a second portion 1612. The first portion 1610 can be configured to be positioned into the space 1602 separating the first bone 1604 from the second bone 1606. The second portion 1612 can be configured to extend out of the space 1602 separating the first bone 1604 from the second bone 1606.

For the illustrated embodiment, the seeker 1600 is a wire. Though in other embodiments other types of devices having a first portion, which is configured to be positioned into the space 1602 separating the first bone 1604 from the second bone 1606, and a second portion, which is configured to extend out of the space 1602 separating the first bone 1604 from the second bone 1606, can be used as the seeker 1600. In still other examples, a seeker that is integrally attached to a corresponding guide can be inserted into the joint space.

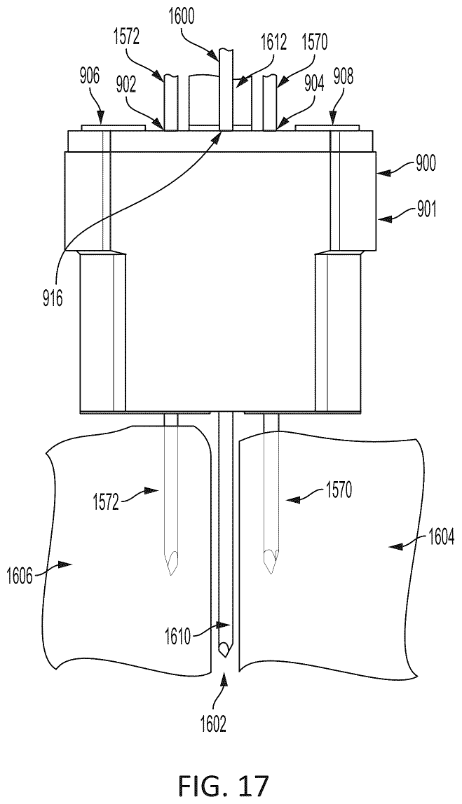

At step 1520, the method 1500 can include aligning a guide using the seeker inserted at step 1510. FIG. 17 is a side elevational view of the guide 900 aligned with the first bone 1604 and/or second bone 1606 using the seeker 1600. Step 1520 can include aligning, using the seeker 1600, a guide that is any one of the guide embodiments disclosed herein, such as the guide 900 or the guide 1000. The guide 900 will be referred to here for ease of reference, though use of the guide 1000 could alternatively be part of step 1520.

As noted, step 1520 can include aligning the guide 900 with the first bone 1604 and/or the second bone 1606 and/or the joint space between the bones using the seeker 1600. The guide 900 can define at least one wire aperture 902, 904 and a seeker receiving aperture 916 at the body 901 of the guide 900. Aligning the guide 900 with the first bone 1604 and/or the second bone 1606 can include: (i) positioning the at least one wire aperture 902, 904 over the first bone 1604 and/or the second bone 1606 and (ii) positioning the seeker receiving aperture 916 of the guide 900 over the second portion 1612 of the seeker 1600 extending out of the space 1602. The second portion 1612 of the seeker 1600 can be received through the seeker receiving aperture 916 and the first portion of 1610 of the seeker 1600 can extend outside of the seeker receiving aperture 916 and be within the space 1602. In this way, by positioning the seeker receiving aperture 916 of the guide 900 over the second portion 1612 of the seeker 1600 that is itself at least partially inserted into the space 1602, the result can be that the at least one wire aperture 902, 904 at the guide 900 is caused to be positioned over at least one of the respective bones 1604, 1606.

In one example, the at least one wire aperture 902, 904 includes a first wire aperture 902 and a second wire aperture 904. In such example, positioning the at least one wire aperture 902, 904 over the first bone 1604 and/or the second bone 1606 can include positioning the second wire aperture 904 over the first bone 1604 and the first wire aperture 902 over the second bone 1606. In this way, by positioning the seeker receiving aperture 916 of the guide 900 over the second portion 1612 of the seeker 1600 that is itself at least partially inserted into the space 1602, the result can be that the second wire aperture 904 is positioned over the first bone 1604 and the first wire aperture 902 is positioned over the second bone 1606. A similar result can occur with respect to the drill guide apertures 906, 908 at the guide 900 by positioning the seeker receiving aperture 916 of the guide 900 over the second portion 1612 of the seeker 1600. Namely, by positioning the seeker receiving aperture 916 of the guide 900 over the second portion 1612 of the seeker 1600 that is itself at least partially inserted into the space 1602, the result can be that the second drill guide aperture 908 is positioned over the first bone 1604 and the first drill aperture 906 can be positioned over the second bone 1606.

The illustrated embodiment shows the seeker 1600 (e.g., a wire) as separable from the guide 900 such that the seeker 1600 can be removably received at the seeker receiving aperture 916 of the guide 900. Though in another embodiment the seeker 1600 can be connected to the guide 900, for instance as a type of keel component at the guide 900. In this integral guide-seeker embodiment, inserting the seeker 1600 into the space 1602 separating the first bone 1604 from the second bone 1606 can include aligning the guide 900 with the first bone 1604 and/or the second bone 1606 using the seeker 1600 connected to the guide 900.

At step 1530, the method 1500 can include inserting wire(s) into bone(s). For example, step 1530 can include inserting a first wire 1570 into the first bone 1604 and a second wire 1572 into the second bone 1606 that is separated from the first bone 1604 by the space 1602. As a more specific such example, step 1530 can include inserting the first wire 1570 into the first bone 1604 and the second wire 1572 into the second bone 1606 using the at least one wire aperture 902, 904. For instance, the first wire 1570 can be inserted into the first bone 1604 through the second wire aperture 904 and the second wire 1572 can be inserted into the second bone 1606 though the first wire aperture 902, as shown at the example of FIG. 17. The first wire 1570 can be inserted into the first bone 1604 through the second wire aperture 904 and the second wire 1572 can be inserted into the second bone 1606 though the first wire aperture 902 after the guide has been aligned using the seeker at step 1520. Accordingly, as noted previously, by positioning the seeker receiving aperture 916 of the guide 900 over the second portion 1612 of the seeker 1600 that is itself at least partially inserted into the space 1602, the result can be that the second wire aperture 904 at the guide 900 is positioned over the first bone 1604 and the first wire aperture 902 at the guide 900 is positioned over the second bone 1606 thus facilitating accurate insertion of the first wire 1570 into the first bone 1604 through the second wire aperture 904 and the second wire 1572 into the second bone 1606 though the first wire aperture 902.

In a further embodiment of the method 1500, a step of drilling one or more implant holes in one or more bones can be included, for instance after the guide has been aligned using the seeker at step 1520 and/or after the wires have been inserted into the bones using the guide at step 1530.

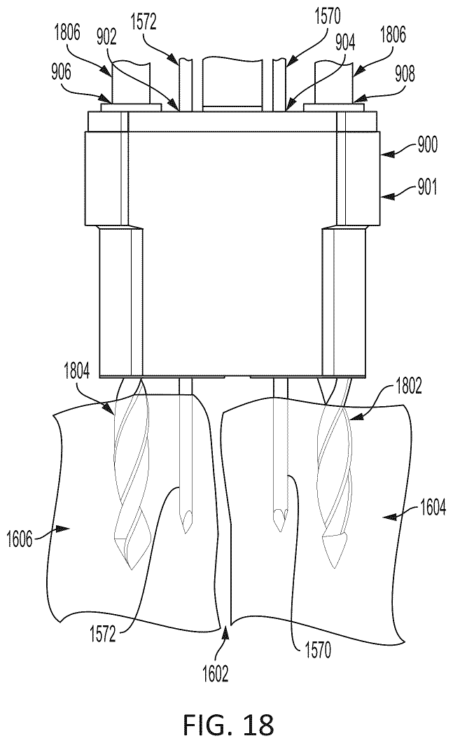

FIG. 18 is a side elevational view of the guide 900 used as a drill guide. In particular, the drill guide 900 can be used to drill a first implant hole 1802 in the first bone 1604 and a second implant hole 1804 in the second bone 1606. A drill member 1806 can be inserted through the second drill guide aperture 908 to drill the first implant hole 1802 in the first bone 1604 and the drill member can be inserted through the first drill guide aperture 906 to drill the second implant hole 1804 in the second bone 1606. The first and second implant holes 1802, 1804 can be drilled using the drill guide 900, for instance, before or after the first wire 1570 is inserted into the first bone 1604 through the second wire aperture 904 and/or when the second wire 1572 is inserted into the second bone 1606 though the first wire aperture 902. The first and second implant holes 1802, 1804 can be drilled, and the drill member size selected, such that the first and second implant holes 1802, 1804 are dimensioned to receive at least a portion of an end portion of a leg of a staple (e.g., at least a portion of the end portion 537 of the leg 502 of the staple 500).

In another, further embodiment of the method 1500, a step of removing the seeker can be included, for instance after the guide has been aligned using the seeker at step 1520, after the wires have been inserted into the bones using the guide at step 1530, and/or after the first and second implant holes 1802, 1804 have been drilled using the drill guide 900. As one such example, the seeker can be removed, from the space separating the first bone from the second bone, and the guide can be removed from the first wire and the second wire prior to aligning the inserter, operatively connected to the implant with the first wire and the second wire, at step 1540.

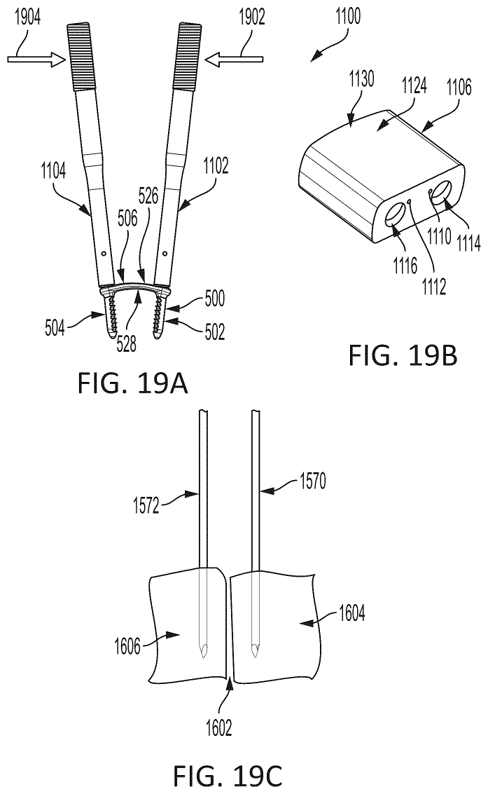

At step 1540, the method 1500 can include aligning an inserter with the wire(s) that were inserted into the bone(s) at step 1530. FIGS. 19A and 19B illustrate components of the inserter 1100 that is to be aligned with wire(s) 1570, 1572 at the first and/or second bones 1604, 1606 illustrated at FIG. 19C. FIG. 19A is an elevational view of the inserter 1100 coupled to a top side of an implant, which in the illustrated example is staple 500. FIG. 19B is a perspective view of a connector 1106, which in this example is a component of the inserter 1100, shown in isolation. FIG. 19C is an elevational view of wires 1570, 1572 at the first and second bones 1604, 1606 for use in aligning the inserter 1100. Step 1540 can include aligning, with the wires 1570, 1572 that were inserted into the bones 1604, 1606 at step 1530, an inserter that is any one of the inserter embodiments disclosed herein, such as the inserter 1100, the inserter 1200, or the inserter 1300. The inserter 1100 will be referred to here for ease of reference, though use of inserter 1200 or 1300 could alternatively be part of step 1540.

The inserter aligned at step 1540 can be operatively connected to an implant, such as the staple 500 as shown at FIG. 19A. Step 1540 can include aligning the inserter 1100, operatively connected to the implant, such as the staple 500, with the first wire 1570 and the second wire 1572 by at least positioning the first wire 1570 in a first wire receiving opening 1110 of the connector 1106 of the inserter 1100 and the second wire 1572 in a second wire receiving opening 1112 of the connector 1106 of the inserter 1100.

In the illustrated example where the implant is the staple 500, the implant can include the first leg 502, the second leg 504, and the bridge 506 connecting the first and second legs 502, 504. The staple can further include the top surface 526 and the bottom surface 528, and the inserter, such as via the first and second coupling shafts 1102, 1104, can be operatively connected to the staple 500 at a location on the staple 500 spaced apart from the bottom surface 528. For instance, the inserter 1100 can be operatively connected to the staple 500 at a location on the staple 500 between the top surface 526 and the bottom surface 528. More particularly, the first coupling shaft 1102, of the inserter 1100, can be connected to the first leg 502 of the staple 500 at a location between the bottom surface 528 and the top surface 526 and the second coupling shaft 1104, of the inserter 1100, can be connected to the second leg 504 of the staple 500 at a location between the bottom surface 528 and the top surface 526.

When aligning the inserter 1100 with the wires 1570, 1570 at step 1540, in addition to the inserter 1100 being operatively coupled to the staple 500, the inserter 1100 can have the connector 1106 joining the first and second coupling shafts 1102, 1104. For example, prior to positioning the first wire 1570 in a first wire receiving opening 1110 of the connector 1106 and the second wire 1572 in a second wire receiving opening 1112 of the connector 1106, the connector 1106 can be connected to the first and second coupling shafts 1102, 1104 so as to join the first and second coupling shafts 1102, 1104. For the illustrated embodiment of the inserter 1100, the connector 1106 can connect to the first and second coupling shafts 1102, 1104, and thereby join the first and second coupling shafts 1102, 1104, by receiving the first coupling shaft 1102 at the first receptacle 1114 at the connector 1106 and receiving the second coupling shaft 1104 at the second receptacle 1116 at the connector 1106.

Connecting the connector 1106 to the first and second coupling shafts 1102, 1104 and to thereby join the first and second coupling shafts 1102, 1104 can cause a load force to be applied at the staple 500 that is operatively connected to the inserter 1100. For example, when the staple 500 is in its natural state, an example of which is shown for the staple 500 at FIG. 19A, and the first and second coupling shafts 1102, 1104 are operatively connected to the staple 500, the first and second coupling shafts 1102, 1104 can be oriented at a skewed angle relative to one another, an example of which is shown for the first and second coupling shafts 1102, 1104 at FIG. 19A. Then, connecting the connector 1106 to the first and second coupling shafts 1102, 1104 can apply the load force to cause the first coupling shaft 1102 to move in a direction 1902 and second coupling shafts 1104 to move in a direction 1904 thereby moving the first and second coupling shafts 1102, 1104 closer together.

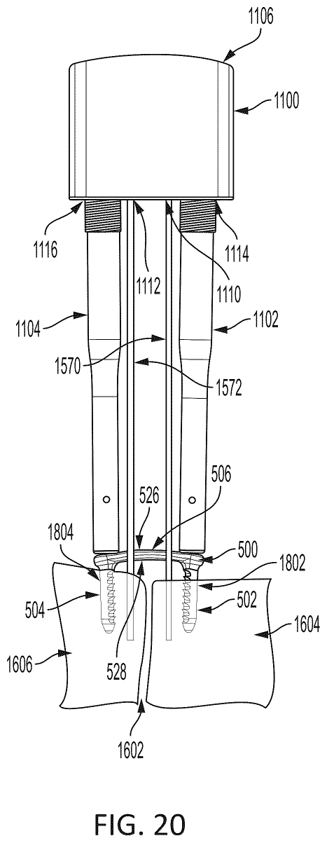

Because the first and second coupling shafts 1102, 1104 are operatively connected to the staple 500, this movement of the first coupling shaft 1102 in the direction 1902 and the second coupling shafts 1104 in the direction 1904 can transfer the load force applied at the first and second coupling shafts 1102, 1104 by the connector 1106 to the staple 500. As a result of this load force being applied at the staple 500, the staple 500 can transition from the natural state, an example of which is shown for the staple 500 at FIG. 19A, to the deformed insertion state, an example of which is shown for the staple 500 at FIG. 20 when the connector 1106 has been applied to join the first and second coupling shafts 1102, 1104 and apply the load force.

When the staple 500 is in the deformed insertion state, the first and second legs 502, 504 can be brought to a more parallel orientation with respect one another than when in the biased compression-inducing state. The deformed insertion state, with the first and second legs 502, 504 in a generally parallel orientation with respect one another, can be a useful orientation for inserting the staple 500 into the bones 1604, 1606 (e.g., at step 1550), while the biased compression-inducing state, with the first and second legs 502, 504 at skewed angles and generally closer together and pointing toward one another, can be a useful orientation after the staple 500 has been inserted into the bones 1604, 1606 so as to apply a compression force at each bone 1604, 1606 and across the space between the bones 1604, 1606 (e.g., after the staple 500 has been positioned at step 1550; at a step of removing the connector). Accordingly, at step 1540, when the inserter 1100 is aligned with the wires 1570, 1572 (e.g., when the first wire receiving opening 1110 receives the wire 1570 and the second wire receiving opening 1112 receives the wire 1572), the connector 1106 can also receive the first coupling shaft 1102 (e.g., at the receptacle 1114) and the second coupling shaft 1104 (e.g., at the second receptacle 1116) such that the staple 500 is in the deformed insertion state.

At step 1550, the method 1500 can include advancing the inserter, aligned at step 1540, along the wire(s) to position the implant, such as the staple 500. FIG. 20 is a side elevational view of the assembled inserter 1100 advanced along the wires 1570, 1572 to position the staple 500 in contact with the first and second bones 1604, 1606.

As noted, in one example, step 1550 can include advancing the inserter 1100 along the first wire 1570 and the second wire 1572 to position the staple 500 in contact with the first bone 1604 and the second bone 1606 with the bridge 506 of the staple 500 bridging across the space 1602 between the first bone 1604 and the second bone 1606. Such advancing the inserter 1100 along the first wire 1570 and the second wire 1572 to position the staple 500 in contact with the first bone 1604 and the second bone 1606 can include positioning the first leg 502 in the first implant hole 1802 in the first bone 1604 and positioning the second leg 504 in the second implant hole 1804 in the second bone 1606. Such advancing the inserter 1100 along the first wire 1570 and the second wire 1572 to position the staple 500 in contact with the first bone 1604 and the second bone 1606 can also include contacting the first bone 1604 and the second bone 1606 with the bottom surface 528 of the staple 500. In one specific such example, contacting the first bone 1604 and the second bone 1606 with the bottom surface 528 of the staple 500 can include contacting the first bone 1604 generally flush with the bottom surface 528 and contacting the second bone 1606 generally flush with the bottom surface 528.

As noted, when the inserter 1100 has been aligned with the wires 1570, 1572 (e.g., when the first wire receiving opening 1110 receives the wire 1570 and the second wire receiving opening 1112 receives the wire 1572) and the connector 1106 also receives the first coupling shaft 1102 (e.g., at the first receptacle 1114) and the second coupling shaft 1104 (e.g., at the second receptacle 1116)), the staple 500 can be in the deformed insertion state. Likewise, when the inserter 1100 is moved along the wires 1570, 1572 to position the staple 500 at step 1550 the staple 500 can be in the deformed insertion state (e.g., by maintaining the connector 1106 joining the first and second coupling shafts 1102, 1104). As seen at the example deformed insertion state of the staple 500 shown at FIG. 20, when the connector 1106 joins the first and second coupling shafts 1102, 1104 and the inserter 1100 is advanced along the wires 1570, 1572, the first leg 502 can be generally parallel to the second leg 504.

In a further embodiment, the method 1500 can additionally include a step of removing the connector from the inserter. For example, the step of removing the connector from the inserter, when included in the method 1500, can occur after the inserter has been advanced along the wires and the implant has been positioned at the target anatomy at step 1550. FIG. 21 is a side elevational view of the inserter 1100 but with the connector 1106 removed such that the implant (e.g., staple 500) helps to fixate the first and second bones 1604, 1606 and applies a compression force at the first and second bones 1604, 1606.

After advancing the inserter 1100 along the first wire 1570 and the second wire 1572 to position the staple 500 in contact with the first bone 1604 and the second bone 1606, the connector 1106 can be removed from the inserter 1100, thereby removing the load force applied by the connector 1106 and causing the first coupling shaft 1102 and the second coupling shaft 1104 to move away from each other—the first coupling shaft 1102 can be caused to move in a direction 1906 (e.g., opposite the direction 1902 in which the first coupling shaft 1102 is caused to move when the connector 1106 joins the first coupling shaft 1102 to the second coupling shaft 1104) and the second coupling shaft 1104 can be caused to move in a direction 1908 (e.g., opposite the direction 1904 in which the second coupling shaft 1104 is caused to move when the connector 1106 joins the second coupling shaft 1104 to the first coupling shaft 1102). Furthermore, the step of removing the connector 1106 from the inserter 1100, when included in the method 1500, can occur after the first leg 502 is positioned in the first implant hole 1802 of the first bone 1604 and after the second leg 504 is positioned in the second implant hole 1804 of the second bone 1606. In an additional example, the step of removing the connector 1106 from the inserter 1100 can occur after the bottom surface 528 of the staple 500 is positioned in contact with the first bone 1604 and the second bone 1606. And, in another additional example, the step of removing the connector 1106 from the inserter 1100 can occur before the first coupling shaft 1102 and the second coupling shaft 1104 are removed from the operative couplings to the staple 500.

In one example, to help remove the connector 1106 and prior to removing the connector 1106, the first coupling shaft 1102 and the second coupling shaft 1104 can be toward each other (e.g., the first coupling shaft 1102 can be moved in a direction opposite the direction 1906 and the second coupling shaft 1104 can be moved in a direction opposite the direction 1908) to help disengage the first and second coupling shafts 1102, 1104 from the connector 1106. For example, moving the first coupling shaft 1102 and the second coupling shaft 1104 toward each other can help to disengage the first retention feature 1126, when so included at the first coupling shaft 1102, from the first receptacle 1114 (e.g., disengage the first retention feature 1126 from the first retention mating feature 1127 at the first receptacle 1114) and the second retention feature 1128, when so included at the second coupling shaft 1104, from the second receptacle 1116 (e.g., disengage the second retention feature 1128 from the second retention mating feature 1129 at the second receptacle 1116).

When the connector 1106 is removed from the first and second coupling shafts 1102, 1104 that are operatively connected to the staple 500, the first leg 502 of the staple 500 and the second leg 504 of the staple 500 can return toward their natural state as shown in FIG. 21. This can include the first and second legs 502, 504 being in a generally skewed arrangement such that a central longitudinal axis of the leg 502 and a central longitudinal axis of the leg 502 intersect. Moreover, as the connector 1106 is in the process of being removed from the first and second coupling shafts 1102, 1104 that are operatively connected to the staple 500, the first leg 502 and the second leg 504 can incrementally move toward one another as the connector 1106 is being removed. Removing connector 1106 can cause the legs of the staple to return from their deformed position back toward their native position, e.g., with the bones in which the legs of the staple are inserted preventing the legs from returning fully to their native, undeformed position, thereby resulting in a compressive force being applied by the staple across the end faces of the bones.

In some embodiments, more than one implant can be positioned according to the teachings disclosed herein. For example, in one embodiment at least two implants (e.g., two staples) can be positioned in and across the bones 1604, 1606 according to the teachings disclosed herein. This could include, for instance, positioning one staple at a medial cuneiform, a first metatarsal, and across the TMT joint at a dorsal side and positioning another staple at a medial cuneiform, a first metatarsal, and across the TMT joint at a medial side. When positioning at least two such implants in and across the bones 1604, 1606 according to the teachings disclosed herein, the associated technique could further include, prior to positioning such implants at the bones 1604, 1606, making a first incision at a first anatomical location (e.g., adjacent the medial cuneiform, the first metatarsal, and across the TMT joint at the dorsal side) and making a second, different incision at a second, different anatomic location (e.g., adjacent the medial cuneiform, the first metatarsal, and across the TMT joint at the medial side).

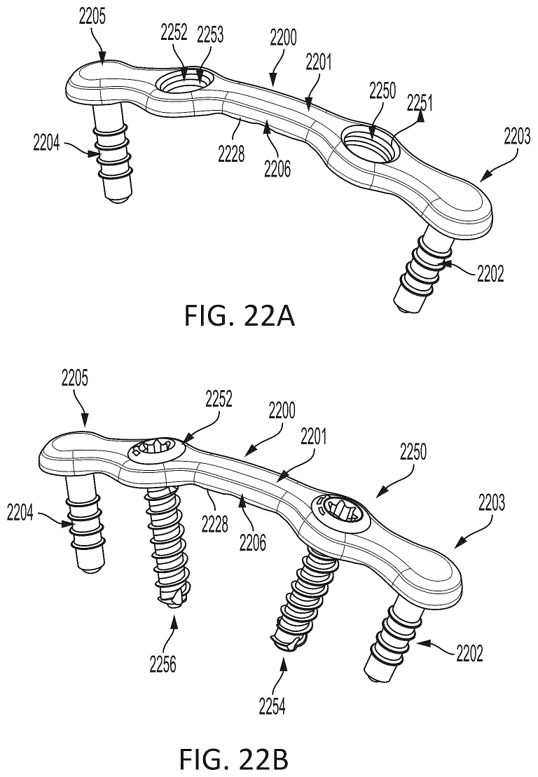

FIGS. 22A-25B illustrate additional embodiments of a staple as one exemplary type of implant that can be used to fixate bones for fusion as described elsewhere herein. In certain embodiments, the staple embodiments illustrated and described with respect to FIGS. 22A-25B can include any one or more features (e.g., each of the features) illustrated and/or described elsewhere herein with respect to orthopedic implants, including with respect to staple embodiments as illustrated and/or described elsewhere herein. The staple embodiments illustrated and described with respect to FIGS. 22A-25B can include one or more fixation apertures, each of which can be configured to receive a fixation member, and, for some embodiments, one or more anatomical contouring features. The presence of one or more fixation apertures and/or one or more anatomical contouring features at a staple can be useful for providing increasing stabilization of the staple at one or more bones when implanted, which can be useful for enhancing the resulting anatomical alignment correction and/or fusion facilitated, at least in part, by implantation of the given staple embodiment.

FIGS. 22A and 22B illustrate an embodiment of an orthopedic implant in the form of a staple 2200 that includes one or more fixation apertures. FIG. 22A is a perspective view of this embodiment of the staple 2200 showing fixation apertures 2250, 2252, and FIG. 22B is a perspective view of this embodiment of the staple 2200 showing fixation members 2254, 2256 placed at the fixation apertures 2250, 2252, respectively. As described elsewhere herein, the staple 2200 can be configured to apply a compression force at the bones and across the space (e.g., joint) between the bones for use in fixating and fusing bones. The staple 2200 can include any one or more features (e.g., each of the features) illustrated and/or described elsewhere herein with respect to orthopedic implants, including with respect to other staple embodiments as illustrated and/or described elsewhere herein.

The staple 2200 can include a staple body 2201 having a first leg 2202, a second leg 2204, and a bridge 2206. For the illustrated embodiment, the staple 2200 includes the first leg 2202 at a first side 2203 of the staple 2200 and the second leg 2204 at a second side 2205 of the staple 2200. In this example, the first side 2203 is opposite the second side 2205. The bridge 2206 can connect the first side 2203 and the second side 2205, and, thus, for example, connect the first leg 2202 and the first fixation aperture 2250 with the second leg 2204 and the second fixation aperture 2252.

As noted, the staple 2200 can further include one or more fixation apertures 2250, 2252. Each of the one or more fixation apertures 2250, 2252 can be configured to receive thereat (e.g., therethrough) a respective fixation member 2254, 2256 (e.g., a bone screw). The fixation aperture 2250, 2252 can receive the respective fixation member 2254, 2256 such that the respective fixation member 2254, 2256 extends through the fixation aperture 2250, 2252 and out from a bottom surface 2228 of the staple body 2201. To accommodate a respective fixation member 2254, 2256, each fixation aperture 2250, 2252 can include a respective complementary coupling element 2251, 2253 that can be configured to couple to a fixation member. For example, as illustrated here, the fixation aperture 2250 can include first complementary coupling element 2251 in the form of one of male and female threading that is complementary to and configured to couple to the other of male and female threading included at the coupling element 2254, and the fixation aperture 2252 can include second complementary coupling element 2253 in the form of male or female threading that is complementary to and configured to couple to the other of male and female threading included at the coupling element 2256. Accordingly, the fixation aperture 2250 can be configured to receive thereat and couple to fixation member 2254 (e.g., a first bone screw) and fixation aperture 2252 can be configured to receive thereat and couple to fixation member 2256 (e.g., a second bone screw). Depending on the orientation of the fixation apertures 2250, 2252 relative to the body 2201, the staple 2200 can be configured such that the fixation member 2254 extends parallel to the leg 2202 and the fixation member extends parallel to the leg 2204 or such that the fixation member 2254 extends at a skewed orientation relative to the leg 2202 and the fixation member 2256 extends at a skewed orientation relative to the leg 2204.

The staple 2200 as illustrated at FIGS. 22A and 22B includes the fixation apertures 2250, 2252 inside of (e.g., relative to a longitudinal length of the staple 2200) the legs 2202, 2204. In particular, the staple 2200 can have the leg 2202 and the fixation aperture 2250 at the first side 2203 with the fixation aperture 2250 closer to the bridge 2206 than the leg 2202 as measured along the longitudinal length of the staple 2200, and the staple 2200 can have the leg 2204 and the fixation aperture 2252 at the second side 2205 with the fixation aperture 2252 closer to the bridge 2206 than the leg 2204 as measured along the longitudinal length of the staple 2200. The leg 2202 and the fixation aperture 2250, and thus the first side 2203 of the staple 2200, can be configured to be placed at a first bone and the leg 2204 and the fixation aperture 2252, and thus the second side 2205 of the staple 2200, can be configured to be placed at a second bone (e.g., the second bone different from the first bone or a fragment/piece of the first bone), for instance as disclosed elsewhere herein to apply compression across a space separating the first and second bones.

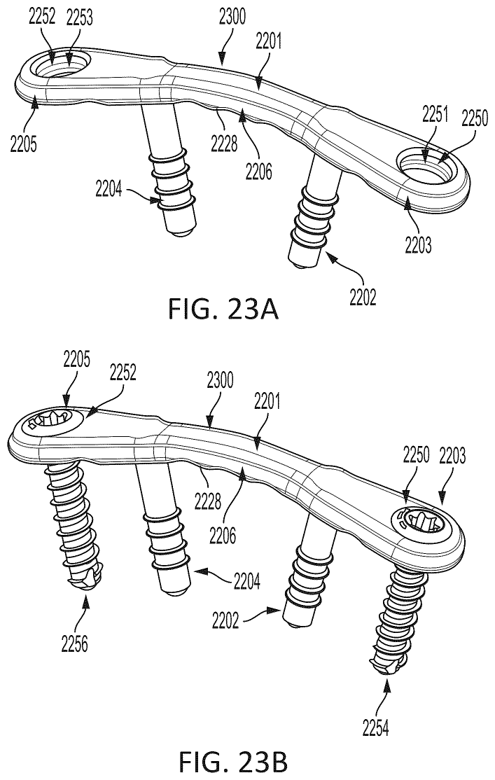

FIGS. 23A and 23B illustrate an additional embodiment of an orthopedic implant in the form of a staple 2300 that includes one or more fixation apertures. The staple 2300 can be similar to, or the same as, the staple 2200 illustrated and described with respect to FIGS. 22A, 22B except as otherwise illustrated and described here with respect to FIGS. 23A, 23B. As such, like reference characters are used to denote like elements. FIG. 23A is a perspective view of this embodiment of the staple 2300 showing the fixation apertures 2250, 2252, and FIG. 23B is a perspective view of this embodiment of the staple 2300 showing fixation members 2254, 2256 placed at the fixation apertures 2250, 2252. The staple 2300 can include any one or more features (e.g., each of the features) illustrated and/or described elsewhere herein with respect to orthopedic implants, including with respect to other staple embodiments as illustrated and/or described elsewhere herein.

Like the staple 2200, the staple 2300 includes the first leg 2202 and the fixation aperture 2250 at the first side 2203 and includes the second leg 2204 and the fixation aperture 2252 at the second side 2205. However, the staple 2300 differs from the staple 2200 in the arrangement of the legs 2202, 2204 and the fixation apertures 2250, 2252. Namely, the staple 2300 as illustrated at FIGS. 23A and 23B includes the fixation apertures 2250, 2252 outside of (e.g., relative to a longitudinal length of the staple 2300) the legs 2202, 2204. In particular, the staple 2300 can have the leg 2202 and the fixation aperture 2250 at the first side 2203 with the leg 2202 closer to the bridge 2206 than the fixation aperture 2250 as measured along the longitudinal length of the staple 2300, and the staple 2300 can have the leg 2204 and the fixation aperture 2252 at the second side 2205 with the leg 2204 closer to the bridge 2206 than the fixation aperture 2252 as measured along the longitudinal length of the staple 2300. The leg 2202 and the fixation aperture 2250, and thus the first side 2203 of the staple 2300, can be configured to be placed at a first bone and the leg 2204 and the fixation aperture 2252, and thus the second side 2205 of the staple 2300, can be configured to be placed at a second bone (e.g., the second bone different from the first bone or a fragment/piece of the first bone), for instance as disclosed elsewhere herein to apply compression across a space separating the first and second bones.

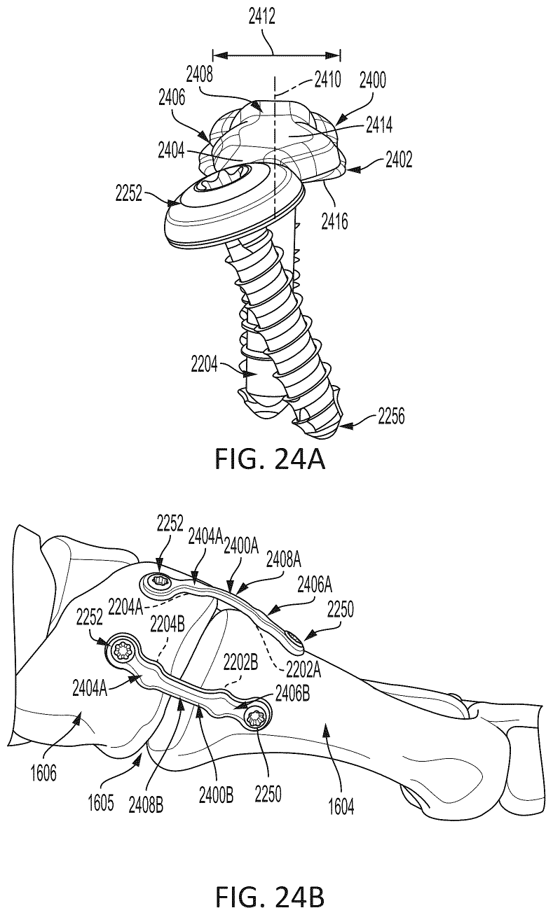

FIGS. 24A and 24B illustrate a further embodiment of an orthopedic implant in the form of a staple 2400 that includes fixation apertures as well as certain exemplary anatomical contouring. FIG. 24A is a side elevational view of this embodiment of the staple 2400 showing exemplary anatomical contouring, and FIG. 24B is a side elevational view showing such staple 2400A, 2400B embodiments with exemplary anatomical contouring placed across a space (e.g., a joint space) separating the first bone 1604 and the second bone 1606. As illustrated at FIG. 24B, staple 2400B can be a mirror image configuration of the configuration of the staple 2400A. The staple 2400 can include any one or more features (e.g., each of the features) illustrated and/or described elsewhere herein with respect to orthopedic implants, including with respect to other staple embodiments as illustrated and/or described elsewhere herein. For example, the staple 2400 can be the same as the staple 2300 but having the anatomical contouring described here with respect to FIGS. 24A and 24B. For certain embodiments, the staple 2400 can include anatomical contouring so as to be an anatomically-fitted staple 2400 similar to, or the same as, that disclosed with respect to anatomically-fitted contouring of implants (e.g., bone plates) in U.S. provisional patent application No. 63/313,162, filed Feb. 23, 2022, and titled “Anatomically-Fitted Tarsometatarsal Bone Plate,” the entire contents of which are incorporated herein by reference.

The staple 2400 can be an anatomically-fitted staple that is geometrically configured to fit the anatomical shape of one or more particular bones. As one example, staple 2400 can be anatomically fitted for a metatarsal fusion procedure such that staple 2400 includes one or more geometric features that are complementary to the anatomical shape of a cuneiform (e.g., medial cuneiform) and/or metatarsal (e.g., first metatarsal).

The staple 2400 can include a body 2402. Body 2402 can include a proximal body region 2404 (2404A of staple 2400A; 2404B of staple 2400B), a distal body region 2406 (2406A of staple 2400A; 2406B of staple 2400B), and a bridge 2408 (2408A of staple 2400A; 2408B of staple 2400B). Proximal body region 2404 can be configured to be positioned over a cuneiform, such as a medial cuneiform as shown as the bone 1606 at FIG. 24B. Distal body region 2406 can be configured to be positioned over a metatarsal, such as a first metatarsal as shown as the bone 1604 at FIG. 24B. Bridge 2408 can extend between proximal body region 2404 and distal body region 2408. Bridge 2408 can be configured to be positioned across a tarsometatarsal joint separating the metatarsal from the cuneiform. Bridge 2408 can define a bridge central longitudinal axis 2410. Body 2402 can have a width 2412 defining an extent of bone plate 2400 transverse to bridge central longitudinal axis 2410. In addition, body 2402 can include a top surface 2414 and a bone facing surface 2416 that is opposite top surface 2414. Staple 2400 can also include fixation holes 2250, 2252 and legs 2202, 2204 as described elsewhere herein, including with fixation hole 2250 and leg 2202 at the distal body region 2406 and the fixation hole 2252 and the leg 2204 at the proximal body region 2404. In general, the body of the staple 2400 may include at least one fixation hole, extending through, and one leg, at, the proximal body region 2404 and at least one fixation hole, extending through, and one leg, at the distal body region 2406.

To help facilitate an anatomical fit of staple 2400 to one or more bones, body 2402 can define staple 2400 as an asymmetric staple. In particular, staple 2400 can be contoured to complement the target anatomy of the one or more bones and/or adjacent joint space(s) over which staple 2400 is to be positioned and fixated. To complement the target anatomy, staple 2400 can include fixation hole 2252 in an asymmetric orientation relative to one or more other fixation holes (e.g., relative to fixation hole 2250). In particular, staple 2400 can include fixation hole 2252, at the proximal body region 2404, in this asymmetric orientation such that fixation hole 2252 complements the native anatomy present at a cuneiform (e.g., present at the medial cuneiform) and/or the joint space between adjacent the cuneiform (e.g., the joint space adjacent the medial cuneiform and the intermediate cuneiform). Fixation hole 2252 can be offset from bridge central longitudinal axis 2410 while fixation hole 2250 is located on bridge central longitudinal axis 2410. More specifically, in some examples, as seen best at FIG. 24B, each of fixation hole 2250, leg 2202B, and leg 2404A can be positioned co-planar with bridge central longitudinal axis 2410 while fixation hole 2252 is offset from bridge central longitudinal axis 2410 one or more planes (e.g., in more than one plane). Thus, the asymmetric orientation of fixation hole 2252 can help to provide staple 2400 as an anatomically-fitted staple that complements the native anatomy present at the cuneiform in way that helps to avoid inadvertent fixation screw placement at a joint space and positions fixation hole 2452 in a manner that accounts for the native anatomy at the cuneiform to facilitate robust fixation screw securement at that native anatomy.

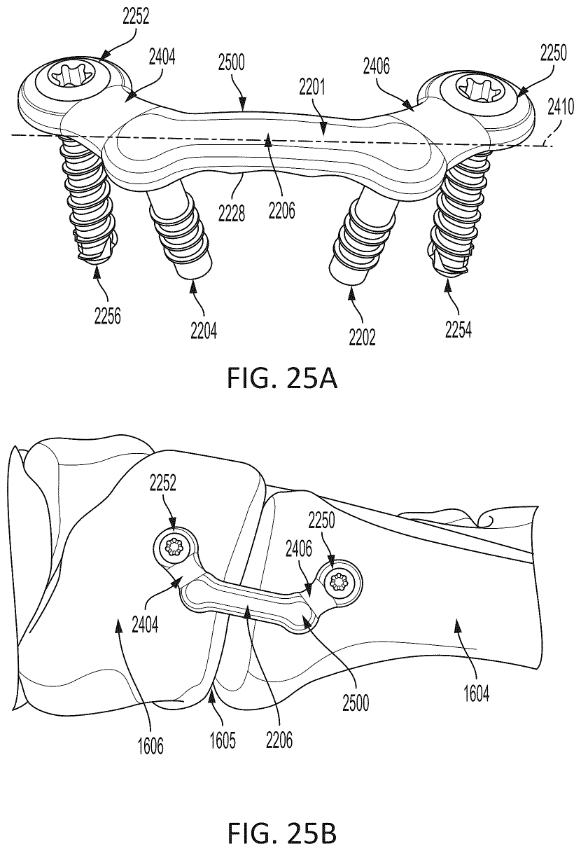

FIGS. 25A and 25B illustrate another embodiment of an orthopedic implant in the form of a staple 2500 that includes fixation apertures as well as certain exemplary anatomical contouring. FIG. 25A is a side elevational view of this embodiment of the staple 2500 showing exemplary anatomical contouring, and FIG. 25B is a side elevational view showing such staple 2500 embodiment with exemplary anatomical contouring placed across a space (e.g., a joint space) 1605 separating the first 1604 and the second bone 1606. The staple 2500 can include any one or more features (e.g., each of the features) illustrated and/or described elsewhere herein with respect to orthopedic implants, including with respect to other staple embodiments as illustrated and/or described elsewhere herein. For example, the staple 2500 can be the same as the staple 2300 but having the anatomical contouring described here with respect to FIGS. 25A and 25B. For certain embodiments, the staple 2500 can include anatomical contouring so as to be an anatomically-fitted staple 2500 similar to, or the same as, that disclosed with respect to anatomically-fitted contouring of implants (e.g., bone plates) in U.S. provisional patent application No. 63/151,041 titled “System and Technique for Metatarsal Realignment with Reduced Incision Length.”

The staple 2500 can be an anatomically-fitted staple that is geometrically configured to fit the anatomical shape of one or more particular bones. As one example, staple 2500 can be anatomically fitted for a metatarsal fusion procedure such that staple 2500 includes one or more geometric features that are complementary to the anatomical shape of a cuneiform (e.g., medial cuneiform) and/or metatarsal (e.g., first metatarsal). The bridge 2206 can be configured to be positioned across the space 1605, such as the tarsometatarsal joint separating the metatarsal from the cuneiform, and the bridge 2206 can define the bridge central longitudinal axis 2410. The illustrated embodiment of the staple 2500 has a generally U-shaped profile to provide anatomical contouring at both the proximal body region 2404, which can be anatomically contoured so as to be configured to fit to the bone 1606 (e.g., the medial cuneiform), and the distal body region 2406, which can be anatomically contoured so as to be configured to fit to the bone 1604 (e.g., the first metatarsal). As such, for the staple 2500, each of the proximal body region 2404 and the distal body region 2406 can diverge from, and be offset from at least in part, the bridge central longitudinal axis 2410. Staple 2500 further includes fixation holes 2250, 2252 and legs 2202, 2204 as described elsewhere herein, including with fixation hole 2250 and leg 2202 at the distal body region 2406 and the fixation hole 2252 and the leg 2204 at the proximal body region 2404. In general, body 2201 may include at least one fixation hole, extending through, and one leg, at, the proximal body region 2404 and at least one fixation hole, extending through, and one leg, at the distal body region 2406.

To help facilitate an anatomical fit of staple 2500 to one or more bones, body 2201 can define staple 2500 as an asymmetric staple. In particular, staple 2500 can be contoured to complement the target anatomy of the one or more bones and/or adjacent joint space(s) over which staple 2500 is to be positioned and fixated. To complement the target anatomy, staple 2500 can include: (i) fixation hole 2250 in an asymmetric orientation relative to the bridge 2206 and/or the leg 2202, and (ii) fixation hole 2252 in an asymmetric orientation relative to the bridge 2206 and/or the leg 2204. In particular, staple 2500 can include fixation hole 2250, at the distal body region 2406, in this asymmetric orientation such that fixation hole 2250 complements the native anatomy present at a metatarsal (e.g., present at the first metatarsal), and staple 2500 can include fixation hole 2252, at the proximal body region 2404, in this asymmetric orientation such that fixation hole 2252 complements the native anatomy present at a cuneiform (e.g., present at the medial cuneiform). To form the generally U-shaped configuration of the body 2201 of the staple 2500, each of fixation hole 2250 and fixation hole 2252 can be offset from bridge central longitudinal axis 2410 (e.g., bridge central longitudinal axis 2410 does not intersect either of fixation hole 2250 or 2252). More specifically, in some examples, each of fixation hole 2250, 2252 can diverge and be offset from the bridge central longitudinal axis 2410 at a same side of the bridge central longitudinal axis 2410. Yet, the staple 2500 can be symmetrical, with respect to each “half” of the U-shape about a radial plane extending radially, and perpendicular to the bridge central longitudinal axis 2410, through a center of the bridge 2206. The anatomic contouring included at the staple 2500 can help to provide staple 2500 as an anatomically-fitted staple that complements the native anatomy present at two or more bones (e.g., the medial cuneiform and the first metatarsal) in way that helps to avoid inadvertent fixation screw placement at a joint space and positions fixation holes 2250, 2252 in a manner that accounts for the native anatomy at the two bones to facilitate robust fixation screw securement at that native anatomy.

A staple configured according to the teachings described herein, when implemented with one or more fixation apertures configured to receive one or more corresponding screws, can have any suitable number or arrangement of legs and bone screws. In general, the staple may have at least one leg on a first side of a bridge and at least one leg on a second side of the bridge, with the two legs separated by the length of the bridge. The staple may have only a single fixation aperture configured to receive a screw (e.g., a fixation aperture configured to be positioned over a metatarsal or a cuneiform with a fixation screw inserted therein without having a fixation aperture positioned over the other bone). The staple may have multiple fixation apertures each configured to receive a screw.

When configured with multiple fixation apertures, the multiple fixation apertures may be positioned on a single side of a bridge of the staple (e.g., providing two or more fixation apertures configured to be positioned over a metatarsal or a cuneiform with fixation screws inserted therein without having a fixation aperture positioned over the other bone). Alternatively, the staple may have at least one fixation aperture on a first side of the bridge and at least one fixation aperture on a second side of the bridge. The relative position of the legs of the staple to the one or more fixation apertures and bridge can vary. In some examples, the staple includes one or more fixation aperture positioned between a leg positioned closest to the bridge and the bridge itself. Additionally or alternatively, the staple may include one or more fixation aperture positioned farther away from the bridge than a leg positioned closest to the bridge.

When configured with one or more fixation apertures, the one or more fixation apertures can be configured to receive a locking screw (e.g., having threading around the head of the screw that screws into corresponding threading surrounding the fixation aperture) or a compression screw (e.g., where the head of the screw is devoid of threading and the fixation aperture does not have threading into which the head of screw is screwed into). If configured with multiple fixation apertures, all the multiple fixation apertures can be configured to receive locking screws, all the multiple fixation apertures can be configured to receive compression screws, or combinations of one or more fixation apertures configured to receive a locking screw and one or more fixation apertures configured to receive a compression screw can be used. The one or more fixation apertures and corresponding screws can be configured as uni-axial in which the screw is configured to be inserted in only a single axial orientation, or the one or more fixation apertures and corresponding screws can be configured as poly-axial in which the screw can be inserted at a selected one of multiple different axial trajectories.

In use, a clinician can prepare, align, and/or insert the one or more legs of the staple into corresponding bone holes as described herein. Before or after placing the legs of the staple into the bone portions, the clinician may drill one or more bone holes into each bone portion underlying the one or more fixation holes of the staple. The clinician can then insert a fixation member (e.g., screw) into each corresponding fixation aperture of the staple and into the underlying bone portion.

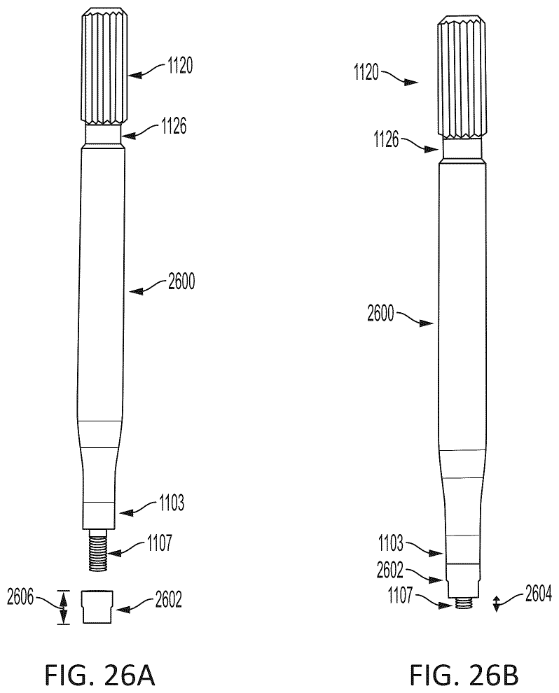

FIGS. 26A-27D illustrate additional embodiments of an inserter coupling shaft that include multi-piece assemblies, for instance for use as a component of an inserter embodiment to place an orthopedic implant as described elsewhere herein. In certain embodiments, the multi-piece inserter coupling shaft embodiments illustrated and described with respect to FIGS. 26A-27D can include any one or more features (e.g., each of the features) illustrated and/or described elsewhere herein with respect to inserter coupling shafts and/or inserters more generally. For example, the inserter coupling shaft embodiments illustrated and described with respect to FIGS. 26A-27D can be useful in facilitating coupling of the inserter coupling shaft embodiments to an implant (e.g., staple) without the inserter coupling shaft embodiments extending out from a bottom surface of the implant so as to help provide a flush contact interface between the implant and the surface(s) of the bone(s) at which the implant is placed using the inserter that includes such coupling shaft embodiments.

FIGS. 26A and 26B illustrate an embodiment of a multi-piece inserter coupling shaft 2600. FIG. 26A is an exploded, elevational view of this inserter coupling shaft 2600 embodiment, and FIG. 26B is an assembled view of this inserter coupling shaft 2600 embodiment. The inserter coupling shaft 2600 can have any one or more features (e.g., each of the features) illustrated and/or described elsewhere herein with respect to inserter coupling shaft embodiments, including features or use as part of an inserter embodiment more generally, except as otherwise described here. As such, like reference characters are used to indicate like elements.

The inserter coupling shaft 2600 can be configured to operatively connect to a connector (e.g., the connector 1106) that is part of an inserter and can selectively join multiple (e.g., two) coupling shafts 2600, as described elsewhere herein. For example, the proximal end portion 1120 of the inserter coupling 2600 can be configured to be removably received at the connector and used with the connector as described elsewhere herein. To help facilitate a more robust removable joining of the coupling shaft 2600 to the connector, the coupling shaft 2600 can include a retention feature 1126. The retention feature 1126 can be located at or near the proximal end portion 1120 of the coupling shaft 2600. The retention feature 126 can be configured to help hold the coupling shaft 2600 in a receptacle at the connector (e.g., in a retention mating feature at the receptacle of the connector), for instance as described elsewhere herein.

The inserter coupling shaft 2600 can be further configured to operatively connect to an implant, such as a staple. In particular, the coupling shaft 2600 can be configured to operatively couple to the staple at a handling coupling at the staple. For example, the coupling shaft 2600 can have the distal end portion 1103 which can include the implant coupling member 1107 (e.g., including threading complementary to threading at a handling coupling of a staple or other type of mechanical connection). The coupling member 1107 at the distal end portion 1103 of the coupling shaft 2600 can be configured to operatively connect to a complementary coupling member at the handling coupling of the staple. In this way, an inserter embodiment can include the coupling shaft 2600 connected to an implant, such as a first side of a staple.

When operatively connected to the staple, the coupling shaft 2600 can be configured to couple to a handling coupling receptacle at the staple at a location between the top surface of the staple and the bottom surface of the staple. For example, the coupling shaft 2600 can operatively couple to a respective handling coupling at a staple from the top surface of the staple and in a direction moving toward the bottom surface of the staple but without the coupling shaft 2600 extending out from the bottom surface of the staple. To help facilitate this coupling, as one such example, the coupling member 1107 at the coupling shaft 2600 can extend within the respective handling coupling receptacle at the staple such that a distal end of the coupling member 1107 at the coupling shaft 2600 is contained within the staple's body (e.g., within the respective handling coupling receptacle at the staple). As such, the coupling member 1107 can extend out from the distal end portion 1103 of the coupling shaft 2600 a distance 2604 that is equal to or less than a depth of the respective handling coupling receptacle at the staple. This can help to facilitate a generally flush contact interface between the bottom surface of the staple and the surface of one or more bones.

To help facilitate this type of generally flush contact interface with the staple or other implant, the coupling shaft 2600 can include a collet 2602 at the distal end portion 1103. The collet 2602 can be attached to the distal end portion 1103 of the coupling shaft 2600 and over a portion (e.g., some but not all) of the coupling member 1107. For instance, where the coupling member 1107 is threaded, the collet 2602 can include complementary threading that is configured to attach to the coupling member 1107. Once the collect 2602 is attached (e.g., threaded) to the coupling member 1107 at a position on the distal end portion 1103 of the coupling shaft 2600 to define the distance 2604 at which the coupling member 1107 extends out from the collet 2602, the collet 2602 can be further attached (e.g., welded, tightened, etc.) to the distal end portion 1103 of the coupling shaft 2600 at that position.

As such, the collect 2602 can be used to help define the extent of the coupling member 1107 from the distal end portion 1103 of the coupling shaft 2600. In other words, by using the collet 2602 of a selected collet length 2606, the inclusion of the collet 2602 at the distal end portion 1103 can cover over some of the extent of coupling member 1107 (e.g., a length of the coupling member equal to the collet length 2606) such that the remaining, exposed portion of the coupling member 1107 is configured to be of an extent that extends out from the distal end portion 1103 of the coupling shaft 2600 the distance 2604 that is equal to or less than the depth of the respective handling coupling receptacle at the staple. As such, the distance 2604 at which the coupling member 1107 extends out from the collet 2602 can be sufficient to accommodate stresses at the staple-to-coupling shaft connection when handling the staple (e.g., energizing the staple) but small enough so that the coupling member 1107 does not extend out from the bottom surface of the staple when the coupling shaft 2600 is coupled to the staple. For example, upon attachment, the bottom surface of collect 2602 can contact the top surface of the implant. As a result, bending forces applied by the inserter may be applied through the contacting surface of the inserter with implant rather than through the threading of the inserter.

FIGS. 27A-27D illustrate another embodiment of a multi-piece inserter coupling shaft 2700. FIG. 27A is an exploded, elevational view of this inserter coupling shaft 2700 embodiment. FIG. 27B is a close-up elevational view of the distal end portion 1103 of the inserter coupling shaft 2700 embodiment. FIG. 27C is an elevational view of this inserter coupling shaft 2700 embodiment coupled to an exemplary orthopedic implant shown here as staple 500. FIG. 27D is a close-up elevational view of the distal end portion 1103 of the inserter coupling shaft 2700 embodiment when coupled to the staple 500 as in FIG. 27C. The inserter coupling shaft 2700 can have any one or more features (e.g., each of the features) illustrated and/or described elsewhere herein with respect to inserter coupling shaft embodiments, including features or use as part of an inserter embodiment more generally, except as otherwise described here. As such, like reference characters are used to indicate like elements.

As shown at the exploded illustration of FIG. 27A, the inserter coupling shaft 2700 can include an inner shaft 2701 and an outer shaft 2702. The inner shaft 2701 can be positioned at least partially inside of the outer shaft 2702. For example, the inner shaft 2701 can include the coupling member 1107 at a distal end of the inner shaft 2701, and the inner shaft 2701 can be positioned within the outer shaft 2702 such that the coupling member 1107 at the inner shaft 2701 extends out from a distal end of the outer shaft 2702. The inner shaft 2701 and the outer shaft 2702 can be rotatably attached to one another such that one of inner and outer shaft 2701, 2702 is configured to rotate relative to the other of the inner and outer shaft 2701, 2702. For example, a user can actuate (e.g., rotate) the proximal end portion 1120 at the inner shaft 2701 to cause the outer shaft 2702 to move in a direction 2704 along a longitudinal axis of the coupling shaft 2700. Thus, in this example, rotating inner shaft 2701 can cause outer shaft 2702 to move closer to the staple 500 (and thus tighten the connection between the staple 500 and the coupling shaft 2700, for instance, when energizing and/or placing the staple 500 at one or more bones) and/or further from the staple 500 (and thus loosen the connection between the staple 500 and the coupling shaft 2700, for instance, to remove the coupling shaft from the staple 500).

As with other coupling shaft embodiments disclosed elsewhere herein, the coupling shaft 2700 can be configured to facilitate a generally flush contact interface with the staple 500 or other implant. As one such example, the coupling shaft 2700 can include the coupling member 1107 that extends out from the distal end portion 1103 of the coupling shaft 2700 the distance 2604 that is equal to or less than a depth of the respective handling coupling receptacle at the staple 500. As such, the inner shaft 2701 can be longer than the outer shaft 2702 at least by the distance 2604 so that when the inner shaft 2701 is attached (e.g., rotatably attached) to the outer shaft 2702, the coupling member 1107 at the inner shaft 2701 extends out the distance 2604 from the outer shaft 2702. This configuration of the coupling shaft 2700 with the extent of the coupling member 1107 at the noted distance 2604 can help to facilitate a generally flush contact interface between the bottom surface of the staple 500 and the surface of one or more bones.

The coupling shaft 2700 can include one or more features to help provide stability during the implant placement process (e.g., including energization of the implant). As one such example, to help provide added stability when applying a load force at the staple 500, the coupling shaft 2700 can include a shaft stabilizing arm 2704. For the illustrated embodiment, the shaft stabilizing arm 2704 is included at a distal end portion the outer shaft 2702 while the coupling member 1107 is included at a distal end portion of the inner shaft 2701 such that when the coupling shaft 2700 is assembled the shaft stabilizing arm 2704 can be adjacent to the coupling member 1107. When so included, the shaft stabilizing arm 2704 can be at the distal end portion 1103 of the coupling shaft 2700 and extend in a direction parallel to a central longitudinal axis of the coupling shaft 2700. Where the implant is a staple, such as the staple 500, the shaft coupling arm 2704 can be configured to contact the bridge 506 of the staple 500 (e.g., at a top and/or side surface of the bridge but not a bottom surface of the bridge facing the bone(s)) when the coupling member 1107 of the coupling shaft 2700 is at the respective handling coupling at the staple, such as shown at FIG. 27C.

As best shown at FIGS. 27B and 27D, the illustrated embodiment of the shaft stabilizing arm 2704 includes a stabilizing arm tab 2706 and a stabilizing arm shoulder 2708. The tab 2706 can be offset and spaced radially apart from the coupling member 1107, and the tab 2706 can extend parallel to a central longitudinal axis of the coupling shaft 2700 (e.g., parallel to a central longitudinal axis of the inner shaft 2701). The tab 2706 can be spaced apart as such from the coupling member 1107 a radial distance to facilitate the tab 2706 contacting the bridge 506 while the coupling member 1107 is at the handling coupling 508 at the staple 500. More specifically, the tab 2706 can be spaced apart as such from the coupling member 1107 a radial distance to facilitate the tab 2706 contacting a side surface 506A of the bridge 506 while the coupling member 1107 is at the handling coupling 508 at the staple 500. The side surface 506A can, at least in part, form a perimeter sidewall of the staple 500 connecting the top surface 526 of the staple 500 to the bottom surface 528 of the staple 500. In this way, the tab 2706 can act to constrain movement of the staple, such as during the staple placement process, and the tab 2706 can be configured to impede or prevent relative rotation at least between the outer shaft 2702 and the staple 500. The shoulder 2708 likewise can be offset and spaced radially apart from the coupling member 1107. The shoulder 2708 can extend generally perpendicular to the central longitudinal axis of the coupling shaft 2700 (e.g., perpendicular to a central longitudinal axis of the inner shaft 2701). The shoulder 2708 can be spaced apart as such from the coupling member 1107 a radial distance to facilitate the shoulder 2708 contacting the bridge 506 while the coupling member 1107 is at the handling coupling 508 at the staple 500. However, while the tab 2706 can be configured to contact the side surface 506A of the bridge 506 while the coupling member 1107 is at the handling coupling 508 at the staple 500, the shoulder 2708 can be configured to contact the top surface 526 of the bridge 506 while the coupling member 1107 is at the handling coupling 508 at the staple 500.

For certain embodiments of the coupling shaft 2700, including the illustrated embodiment at FIGS. 27A-27D, the coupling shaft 2700 can include a second tab 2707. The second tab 2707 can be located at the outer shaft 2702 and radially spaced apart from the coupling member 1107, such as radially spaced apart from the coupling member 1107 at a different side of the coupling member 1107 than the tab 2706. Like the tab 2706, the second tab 2707 can be configured to contact the side surface 506A of the bridge 506, though the second tab 2707 can be configured to contact an opposite side surface 506A of the bridge 506 than the tab 2706. The second tab 2707 can be configured to impede or prevent relative rotation at least between the outer shaft 2702 and the staple 500, including working in cooperation with the tab 2706 such that the tab 2706 and the second tab 2707 are configured to impede opposite directions of relative rotation at least between the outer shaft 2702 and the staple 500.

Notably, the disclosed configuration of the coupling shaft 2700 can leave the bottom surface 528 of the staple 500 unobstructed such that the bottom surface 528 of the staple 500 can sit generally flush at a surface of one or more bones without the coupling shaft 2700 extending out beyond the bottom surface 528 of the staple 500. And, at the same time, the coupling shaft 2700 can include one or more features, such as the shaft stabilizing arm 2704, to help provide placement, energization, and/or insertion stability during the application of one or more forces at the staple 500 (e.g., application of a load force at the staple 500).

As noted, the coupling shaft 2700 can be configured to couple to the staple 500 through the top surface 526 of the staple 500. The coupling shaft 2700 can be configured, for instance, to couple to the first handling coupling 508 and/or the second handling coupling 510 through the top surface 526 of the staple 500 without extending under the bottom surface 528 of the staple 500. In one exemplary application where the staple 500 is to be placed at a metatarsal, at a cuneiform, and bridging across a space (e.g., joint space, such as the TMT joint space) between the metatarsal and cuneiform, when the coupling shaft 2700 is connected to the first handling coupling 508 and/or the second handling coupling 510 through the top surface 526 without extending under the bottom surface 528 of the staple 500, the bottom surface 528 of the staple 500 can be configured to directly contact at least one of a metatarsal and a cuneiform without any inserter structure, including without any coupling shaft 2700 structure, present between the bottom surface 528 and the at least one of the metatarsal and the cuneiform. This configuration can allow the bottom surface 528 of the staple 500 to be more flushly placed at the metatarsal and/or cuneiform as compared to a configuration where an inserter structure, including a structure of the coupling shaft 2700, is present between the bottom surface 528 and the metatarsal and/or cuneiform when the coupling shaft 2700 is coupled to the staple 500.

In some examples where the coupling shaft 2700 is configured to couple to the staple 500 through the top surface 526 of the staple 500 and includes one or more shaft stabilizing arm(s) 2704, the coupling shaft 2700 can further be configured to couple to the first handling coupling 508 and/or the second handling coupling 510 through the top surface 526 of the staple 500 without the one or more shaft stabilizing arm(s) 2704 extending down an entire thickness 550 of a perimeter sidewall (e.g., sidewall 506A) of the staple 500 connecting the top surface 526 of the staple 500 to the bottom surface 528 of the staple 500. For example, the bridge 506 can define thickness 550 of the bridge 506, and the coupling shaft 2700 can further be configured to couple to the first handling coupling 508 and/or the second handling coupling 510 through the top surface 526 of the staple 500 without the one or more shaft stabilizing arm(s) 2704 extending down an entire thickness 550 of the bridge 506. In an alternate example where the coupling shaft 2700 is configured to couple to the staple 500 through the top surface 526 of the staple 500, the coupling shaft 2700 can further be configured to couple to the first handling coupling 508 and/or the second handling coupling 510 through the top surface 526 of the staple 500 without the shaft stabilizing arm(s) 2704 extending down more than three-quarters of the perimeter sidewall (e.g., sidewall 506A) of the staple 500 connecting the top surface 526 of the staple 500 to the bottom surface 528 of the staple 500. For example, the coupling shaft 2700 can further be configured to couple to the first handling coupling 508 and/or the second handling coupling 510 through the top surface 526 of the staple 500 without the one or more shaft stabilizing arm(s) 2704 extending down more than three-quarters of the thickness 550 of the bridge 506. In another alternate example where the coupling shaft 2700 is configured to couple to the staple 500 through the top surface 526 of the staple 500, the coupling shaft 2700 can further be configured to couple to the first handling coupling 508 and/or the second handling coupling 510 through the top surface 526 of the staple 500 without the shaft stabilizing arm(s) 2704 extending down more than half of the perimeter sidewall (e.g., sidewall 506A) of the staple 500 connecting the top surface 526 of the staple 500 to the bottom surface 528 of the staple 500. For example, the coupling shaft 2700 can further be configured to couple to the first handling coupling 508 and/or the second handling coupling 510 through the top surface 526 of the staple 500 without the one or more shaft stabilizing arm(s) 2704 extending down more than half of the thickness 550 of the bridge 506. In these noted examples, the thickness 550 of the perimeter sidewall (e.g., sidewall 506A) of the staple 500 connecting the top surface 526 of the staple 500 to the bottom surface 528 of the staple 500, and/or the thickness 550 of the bridge 506, can be at least 0.5 mm. As such, in the example where the coupling shaft 2700 is configured to couple to the first and/or second handling couplings 508, 510 without the shaft stabilizing arm(s) 2704 extending down more than three-quarters of the perimeter sidewall (e.g., sidewall 506A) of the staple 500, the coupling shaft 2700 can be configured to so couple without the shaft stabilizing arm(s) 2704 extending down more than three-quarters of the of the at least 0.5 mm thickness 550 at the perimeter sidewall (e.g., sidewall 506A) of the staple 500. And in the example where the coupling shaft 2700 is configured to couple to the first and/or second handling couplings 508, 510 without the stabilizing arm(s) 2704 extending down more than half of the perimeter sidewall (e.g., sidewall 506A) of the staple 500, the coupling shaft 2700 can be configured to so couple without the shaft stabilizing arm(s) 2704 extending down more than half of the of the at least 0.5 mm thickness 550 at the perimeter sidewall (e.g., sidewall 506A) of the staple 500.

Referring to the example shown at FIG. 27D, the coupling shaft 2700 can be configured to couple to the staple 500 through the top surface 526 of the staple 500 such that only the staple 500, and no portion of the inserter (e.g., no portion of the coupling shaft 2700), defines a contact interface with the one or more bones at which the staple 500 is being inserted into. This coupled configuration of the staple 500 and coupling shaft 2700 that results in staple only contact at the one or more bones at which the staple 500 is being inserted into can help to insert the staple 500 flushly at the one or more bones because no inserter (e.g., coupling shaft 2700) structure is present at a location to contact the one or more bones which would prevent flush insertion of the staple 500 at the one or more bones. As one example, the inserter (e.g., coupling shaft 2700) can be configured to couple to the staple 500 by contacting the staple 500 at only locations above the bottom surface 528 of the staple 500. For the illustrated embodiment of the coupling shaft 2700, as best seen at FIG. 27D, the coupling shaft 2700 is configured to contact the staple 500 at only locations above the bottom surface 528 of the staple 500. As shown at this illustrated embodiment at FIGS. 27B and 27D, when the coupling shaft 2700 is coupled to the staple 500, the implant coupling member 1107 and the shaft stabilizing arm 2704 (e.g., including the stabilizing arm tab 2706) contact the staple 500 at only staple portions above the bottom surface 528 of the staple 500—e.g., the implant coupling member 1107 contacts the staple 500 at the handling coupling 508 of the staple 500 only at locations at the handling coupling 508 above the bottom surface 528 of the staple 500 and the shaft stabilizing arm 2704 (e.g., including the stabilizing arm tab 2706) contacts the staple 500 at the sidewall 506a of the staple 500 only at locations at the sidewall 506a above the bottom surface 528 of the staple 500.

For embodiments where the coupling shaft 2700 includes one or more shaft stabilizing arm(s) 2704, the one or more shaft stabilizing arm(s) 2704 can contact the staple 500 along only a single plane. For example, as seen at the illustrated example at FIG. 27D, the sidewall 506a of the staple 500 can define one plane and the shaft stabilizing arm 2704 can contact the sidewall 506a of the staple 500 at only that one plane defined by the sidewall 506a of the staple 500. In such example, the sidewall 506a of the staple 500 can define a planar staple contact surface and the shaft stabilizing arm 2704 can define a planar arm contact surface at the tab 2706, and the planar staple contact surface at the sidewall 50a and the planar arm contact surface at the tab 2706 can define the shaft stabilizing arm 2704 contact with the staple 500 along only the single plane. For instance, in certain embodiments, the only contact between the staple 500 and the coupling shaft 2700 both (i) below the top surface 526 of the staple 500 and (ii) at the outer perimeter of the staple 500 can be the contact between the coupling shaft 2700 and the staple 500 in the single plane.

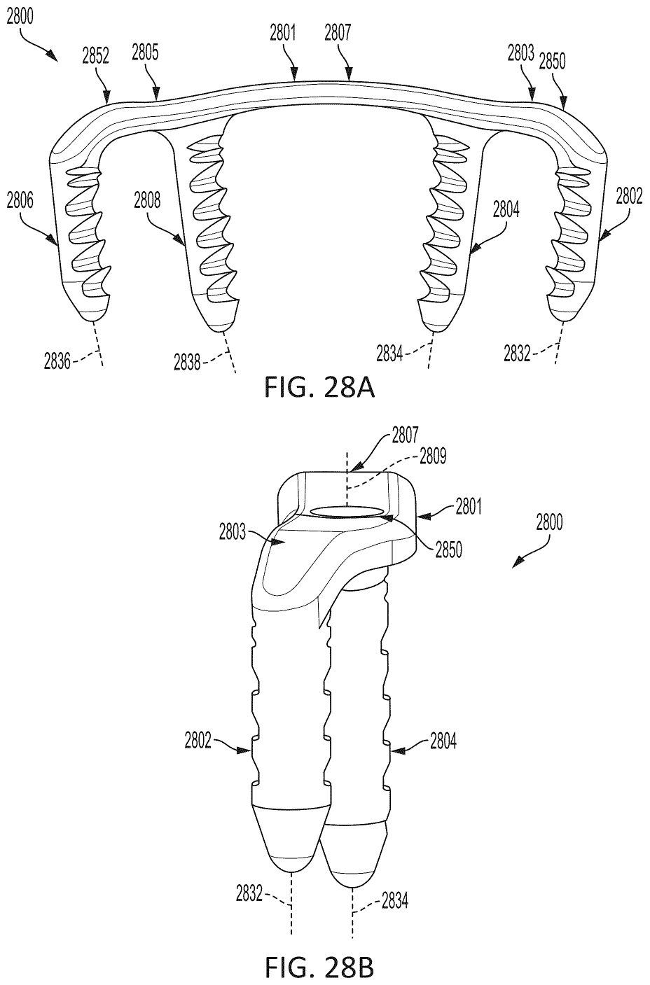

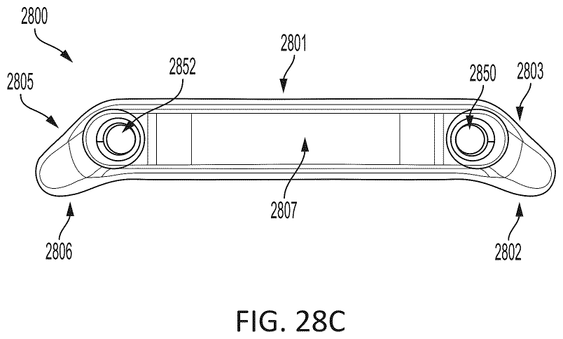

FIGS. 28A-28C show another embodiment of an orthopedic implant in the form of a staple 2800. In particular, FIG. 28A is a longitudinal side elevational view of the staple 2800, FIG. 28B is a radial side elevational view of the staple 2800, and FIG. 28C is a top plan view of the staple 2800.

Aspects of the staple 2800 can be similar to, or the same as, the staple 500 as disclosed elsewhere herein except as otherwise noted here. For example, the staple 2800 can include one or more (e.g., each) of the features disclosed herein with respect to the staple 500 except as otherwise noted here. The staple 2800 is illustrated at FIGS. 28A-28C in an exemplary biased compression-inducing state of the staple 2800. And the staple 2800 can be configured to transition between the biased compression-inducing state and the deformed insertion state as disclosed elsewhere herein (e.g., with respect to the staple 500).

The staple 2800 can include a body 2801 that includes a first side 2803 and a second side 2805 that is opposite the first side 2803. A bridge 2807 at the body 2801 extends between the first side 2803 and the second side 2805. At the body 2801, the staple 2800 includes a first leg 2802, a second leg 2804, a third leg 2806, and a fourth leg 2808. The staple 2800 includes the first leg 2802 and the second leg 2804 at the first side 2803 of the bridge 2807 and the third leg 2806 and the fourth leg 2808 at the second side 2805 of the bridge 2807. The staple 2800 can be symmetrical about the bridge 2807. For example, as shown at FIG. 28A, the staple 2800 can include curvature along the body 2801 at the first side 2803 of the bridge 2807, resulting in one or more elevational changes along the body 2801 at the first side 2803, that is symmetrical to curvature along the body 2801 at the second side 2805 of the bridge 2807, resulting in one or more elevational changes along the body 2801, at the at the second side 2805, that are symmetrical to those one or more elevational changes at the first side 2803. As another additional or alternative example of symmetry defined by the staple 2800, the configuration of the first and second legs 2802, 2804 at the first side 2803 of the staple 2800 can be symmetrical to the configuration of the third and fourth legs 2806, 2808 at the second side 2805 of the staple 2800. As a further additional or alternative example of symmetry defined by the staple 2800, the staple 2800 can be symmetrical about a radial plane, which extends perpendicular to a central longitudinal axis 2809 of the bridge 2807 through a center of the bridge 2807 so as to bisect the staple 2800 at the center of the bridge 2807 with one bisected half including the first side 2803 and the other bisected half including the second side 2805, such that the one bisected half that includes the first side 2803 is symmetrical to the other bisected half that includes the second side 2805.

The symmetrical configuration nature of the staple 2800 at the first and second ends 283, 2805 can be useful in helping to reduce the size of an access incision needed to implant the staple 2800. Also, the symmetrical configuration nature of the staple 2800 can be useful in allowing two of the same configuration staple 2800 to be used at two different locations along one or more bone portions and thereby reduce inventory costs.

As shown for the illustrated embodiment, the staple 2800 can include an offset leg arrangement, of the first and second legs 2802, 2804, at the first side 2803 and an offset leg arrangement, of the third and fourth legs 2806, 2808, at the second side 2805. As seen at FIG. 28B, the first leg 2802 can be offset from the second leg 2804 relative to the central longitudinal axis 2809 of the bridge 2807. For example, the second leg 2804 can be aligned with the central longitudinal axis 2809 of the bridge 2807 while the first leg 2802 can be offset from the central longitudinal axis 2809 of the bridge 2807. And this second leg 2804, which is aligned with the central longitudinal axis 2809, can be closer to the bridge 2807 than the first leg 2802, which if offset from the central longitudinal axis 2809. Likewise, the third and fourth legs 2806, 2808 can have the same, or similar, offset configuration relative to the central longitudinal axis 2809 as the first and second legs 2802, 2804. Namely, in this example, the fourth leg 2808 can be aligned with the central longitudinal axis 2809 of the bridge 2807 while the third leg 2806 can be offset from the central longitudinal axis 2809 of the bridge 2807. And this fourth leg 2808, which is aligned with the central longitudinal axis 2809, can be closer to the bridge 2807 than the third leg 2806, which if offset from the central longitudinal axis 2809.

The staple 2800 can have two or more of the legs 2802, 2804, 2806, 2808 that extend in a parallel orientation relative to one another. For example, the first leg 2802 and the second leg 2804 extend parallel to one another, and the third leg 2806 and the fourth leg extend parallel to one another. The illustrated embodiment shows the relative parallel orientation of the first and second legs 2802, 2804 and the relative parallel orientation of the third and fourth legs 2806, 2808 when the staple 2800 is in the biased compression-inducing state. In particular, the first leg 2802 can define a first leg central longitudinal axis 2832 and the second leg 2804 can define a second leg central longitudinal axis 2834, and the first leg central longitudinal axis 2832 can be parallel to the second leg central longitudinal axis 2834. Likewise, the third leg 2806 can define a third leg central longitudinal axis 2836 and the fourth leg 2808 can define a fourth leg central longitudinal axis 2838 (e.g., when the staple 2800 is in the biased compression-inducing state), and the third leg central longitudinal axis 2836 can be parallel to the fourth leg central longitudinal axis 2838 (e.g., when the staple 2800 is in the biased compression-inducing state). It can also be the case, in a further example, that the angle at which the first leg 2802 extends is inverse to the angle at which the third leg 2806 extends, and the angle at which the second leg 2804 extends is inverse to the angle at which the fourth leg 2808 extends. The first leg 2802 can have a first leg length in a direction extending along the first leg central longitudinal axis 2832 and the second leg 2804 can have a second leg length in a direction extending along the second leg longitudinal axis 2834, with the first leg length being different (e.g., less, as shown for the illustrated embodiment) than the second leg length. Likewise, the third leg 2806 can have a third leg length in a direction extending along the third leg longitudinal axis 2836 and the fourth leg 2808 can have a fourth leg length in a direction extending along the fourth leg longitudinal axis 2836, where the third leg length is different (e.g., less, as shown for the illustrated embodiment) than the fourth leg length. Thus, the outer legs—the first leg 2802 and the third leg 2806—can have a shorter leg length than the inner legs—the second leg 2804 and the fourth leg 2808.

The staple 2800 can also include one or more handling couplings 2850, 2852. As shown for the illustrated embodiment, the staple 2800 includes a first handling coupling 2850 at the first side 2803 and a second handling coupling 2852 at the second side 2805. The first handling coupling 2850 can be located between the first and second legs 2802, 2804, and the second handling coupling 2852 can be located between the third and fourth legs 2806, 2808. In the illustrated embodiment, the handling couplings 2850, 2852 extend all the way through a thickness of the body 2801 such that each handling coupling 2850, 2852 is open at each of the top and bottom sides of the body 2801. Though in other embodiments one or both of the handling couplings 2850, 2852 may only extend partially through the thickness of the body 2801 (e.g., and open at only the top side of the body 2801). As disclosed elsewhere herein, each of the handling couplings 2850, 2852 can be configured to receive, and couple to, a coupling shaft to facilitate, for instance, energization (e.g., to transition the staple 2800 from the biased compression-inducing state to the deformed insertion state) of the staple 2800. As one such example, as disclosed elsewhere herein, each handling coupling 2850, 2852 can include a connection mechanism (e.g., threading) that is complementary to a corresponding connection mechanism (complementary threading) at the respective coupling shaft to facilitate coupling between the respective handling coupling and coupling shaft.

In some examples, in the biased compression-inducing state of the staple 2800 shown at FIGS. 28A-28C, the bridge 2807 can arch upward away from legs 2802, 2804, 2806, 2808 such that for embodiments where the legs 2802, 2804, 2806, 2808 are of equal length, end portions of the legs opposite the bridge 2807 can be at two or more different elevation. For instance, when the staple 2800 is in the biased compression-inducing state, the first leg 2802 and the third leg 2806 (e.g., the outermost legs) terminate at a first, same elevation, while the second leg 2804 and the fourth leg 2808 (e.g., the innermost legs) terminate a second, same elevation that is different than the first elevation (e.g., the second and fourth legs 2804, 2808 terminate at a location further radially away from the bridge 2807 than the first and third legs 2802, 2806).

As disclosed elsewhere herein, the bridge 2807 can have a length that is sufficient to position the bridge across a space (e.g., joint) between bone portions (e.g., two different bones) in the foot while maintaining the first and second legs 2802, 2804 at one such bone portion and the third and fourth legs 2806, 2808 at another such bone portion.

A staple as described herein may be used alone or in combination with one or other bone fixation devices to fixate a joint between opposed bone portions for fusion. Other types of bone fixation devices that can be used include, but are not limited to, a bone screw (e.g., a compressing bone screw), a bone plate, an external fixator, a pin (e.g., an intramedullary implant), and/or combinations thereof. A staple according to the disclosure can be attached before or after installing the one or more other bone fixation devices (when used) to the bone portions being fixated.

Various examples have been described. These and other examples are within the scope of the following claims.

1. A method of fixating bones for fusion, the method comprising:
positioning a first leg of a staple connected to an inserter in a first implant hole of a first bone and a second leg of the staple connected to the inserter in a second implant hole of a second bone, wherein the first leg of the staple is connected to the second leg of the staple by a bridge, wherein the staple is positioned such that a bottom surface of the staple faces the first bone and the second bone and a top surface of the staple faces away from the first bone and the second bone, wherein the inserter is connected to the staple through the top surface of the staple without extending under the bottom surface of the staple; and
detaching the inserter from the staple.

2. The method of claim 1, further comprising:
prior to positioning the first leg of the staple at the first implant hole and the second leg of the staple at the second implant hole, (i) connecting the inserter to a first side of the staple having the first leg through the top surface of the first side of the staple without extending under the bottom surface of the first side of the staple and (ii) connecting the inserter to a second side of the staple having the second leg through the top surface of the second side of the staple without extending under the bottom surface of the second side of the staple.

3. The method of claim 2, wherein the inserter is connected to the first side of the staple through the top surface of the first side of the staple without extending under the bottom surface of the first side of the staple and without contacting an outer perimeter of the first side of the staple, and wherein the inserter is connected to the second side of the staple through the top surface of the second side of the staple without extending under the bottom surface of the second side of the staple and without contacting an outer perimeter of the second side of the staple.

4. The method of claim 1, further comprising:
applying a load force at the staple using the inserter, wherein, as the load force is applied at the staple using the inserter, the first leg and the second leg are caused to move apart from one another.

5. The method of claim 4, further comprising:
removing the load force from the staple (i) after positioning the first leg in the first implant hole and the second leg in the second implant hole and (ii) prior to detaching the inserter from the staple.

6. The method of claim 5, wherein removing the load force from the staple causes the first leg and the second leg to move toward one another to apply a compression force at the first bone and the second bone.

7. The method of claim 6, wherein the inserter comprises a first coupling shaft connected through the top surface of the staple without extending under the bottom surface of the staple and a second coupling shaft connected through the top surface of the staple without extending under a bottom surface of the staple, and wherein applying the load force comprises biasing the first coupling shaft and the second coupling shaft toward each other.

8. The method of claim 7, wherein detaching the inserter from the staple comprises detaching, from the top surface, the first coupling shaft and the second coupling shaft from the staple.

9. The method of claim 8, wherein detaching the first coupling shaft and the second coupling shaft from the staple causes the first coupling shaft to move away from the second coupling shaft, thereby removing the load force and causing the first leg of the staple in the first implant hole and the second leg of the staple in the second implant hole to apply the compression force in a direction toward one other.

10. The method of claim 7, wherein the first coupling shaft is connected through the top surface of the staple over the first leg of the staple and the second coupling shaft is connected through the top surface of the staple over the second leg of the staple.

11. The method of claim 7, wherein a connector joins the first coupling shaft and the second coupling shaft to bias the first coupling shaft and the second coupling shaft toward each other and maintain the load force at the staple while the connector joins the first coupling shaft and the second coupling shaft.

12. The method of claim 11, further comprising:
removing the connector from the first coupling shaft and the second coupling shaft to cause the first coupling shaft to move away from the second coupling shaft.

13. The method of claim 12, wherein removing the connecting causes the load force at the staple to be removed.

14. The method of claim 12,
wherein, when the connector joins the first coupling shaft and the second coupling shaft, the first leg of the staple is oriented generally parallel to the second leg of the staple, and
wherein, when the connector is removed, the first leg of the staple and the second leg of the staple are oriented toward one another.

15. The method of claim 12, wherein the connector comprises a first receptacle and a second receptacle, wherein the first coupling shaft includes a first retention feature and the second coupling shaft includes a second retention feature, and wherein the connector joins the first coupling shaft and the second coupling shaft at least by holding the first retention feature of the first coupling shaft in the first receptacle and holding the second retention feature of the second coupling shaft in the second receptacle.

16. The method of claim 15, further comprising:
prior to removing the connector from the first coupling shaft and the second coupling shaft, moving the first coupling shaft toward the second coupling shaft to disengage the first retention feature from the first receptacle and moving the second coupling shaft toward the first coupling shaft to disengage the second retention feature from the second receptacle.

17. A method of fixating bones for fusion, the method comprising:
positioning a first leg of a staple connected to an inserter in a first implant hole of a first bone and a second leg of the staple connected to the inserter in a second implant hole of a second bone, wherein the first leg of the staple is connected to the second leg of the staple by a bridge, and the inserter comprises a first coupling shaft connected to the first leg of the staple, a second coupling shaft connected to the second leg of the staple, and a connector, with the first coupling shaft and the second coupling shaft being biased toward each other to apply a load force to the staple and the connector joining the first coupling shaft and the second coupling shaft to maintain the load force applied to the staple while the connector joins the first coupling shaft and the second coupling shaft; and
removing the connector from the inserter, thereby removing the load force and causing the first coupling shaft and the second coupling shaft to move apart from one another.

18. The method of claim 17, wherein, when the connector joins the first coupling shaft and the second coupling shaft, the first leg of the staple is oriented generally parallel to the second leg of the staple.

19. The method of claim 18, wherein, when the connector is removed, the first leg of the staple and the second leg of the staple are configured to apply a compression force in a direction toward one another.

20. The method of claim 17, wherein, as the connector is being removed, the first leg of the staple and the second leg of the staple move toward one another.

21. The method of claim 20, wherein the connector is removed after the first leg of the staple is positioned in the first implant hole of the first bone and after the second leg of the staple is positioned in the second implant hole of the second bone.

22. The method of claim 21, wherein the connector is removed after a bottom surface of the staple is positioned in contact with the first bone and the second bone.

23. The method of claim 22, wherein the first coupling shaft is connected to the first leg of the staple at a location between the bottom surface of the staple and a top surface of the staple and the second coupling shaft is connected to the second leg of the staple at a location between the bottom surface of the staple and the top surface of the staple, and wherein the first coupling shaft and the second coupling shaft are removed from the staple after the connector is removed from the inserter.

24. A method of fixating bones for fusion, the method comprising:
inserting a first wire into a first bone and a second wire into a second bone, the first bone being separated from the second bone by a space;
aligning an inserter operatively connected to an implant with the first wire and the second wire by at least positioning the first wire in a first wire receiving opening of the inserter and the second wire in a second wire receiving opening of the inserter; and
advancing the inserter along the first wire and the second wire to position the implant in contact with the first bone and the second bone with the implant bridging between the first bone and the second bone.

25. The method of claim 24, wherein the implant is a staple comprising a first leg and a second leg separated from each other by a bridge, and wherein advancing the inserter along the first wire and the second wire to position the implant in contact with the first bone and the second bone comprises positioning the first leg of the staple in a first implant hole in the first bone and positioning the second leg of the staple in a second implant hole in the second bone.

26. The method of claim 24, wherein:
the inserter comprises a first coupling shaft connected to a first side of the implant, a second coupling shaft connected to a second side of the implant, and a connector;
the first coupling shaft and the second coupling shaft are biased toward each other to apply a load force to the implant; and
the connector joins the first coupling shaft and the second coupling shaft.

27. The method of claim 26, wherein the connector defines the first wire receiving opening and the second wire receiving opening.

28. The method of claim 26, further comprising:
after advancing the inserter along the first wire and the second wire to position the implant in contact with the first bone and the second bone, removing the connector, thereby causing the first coupling shaft and the second coupling shaft to move away from each other.

29. The method of claim 28,
wherein the connector comprises a first receptacle and a second receptacle, wherein the first coupling shaft includes a first retention feature and the second coupling shaft includes a second retention feature, and wherein the first retention feature holds the first coupling shaft in the first receptacle and the second retention feature holds the second coupling shaft in the second receptacle, and
further comprising: prior to removing the connector, moving the first coupling shaft and the second coupling shaft toward each other to disengage the first retention feature from the first receptacle and the second retention feature from the second receptacle.

30. The method of claim 24, further comprising:
aligning a drill guide with the first bone and the second bone such that the drill guide is centered relative the space separating the first bone from the second bone, the drill guide defining at least one wire aperture, wherein aligning the drill guide with the first bone and/or the second bone comprises positioning the at least one wire aperture over the first bone and/or the second bone;
inserting the first wire into the first bone and the second wire into the second bone using the at least one wire aperture; and
drilling a first implant hole in the first bone using the drill guide and a second implant hole in the second bone using the drill guide.

31. The method of claim 24, wherein the implant comprises a top surface and a bottom surface, wherein the inserter is operatively connected to the implant at a location on the implant between the top surface and the bottom surface, and wherein advancing the inserter along the first wire and the second wire to position the implant in contact with the first bone and the second bone comprises contacting the first bone flush with the bottom surface and contacting the second bone flush with the bottom surface.
